(19) United States
(12) Patent Application Publication
(10) Pub. No.: US 2020/0400659 A1
(43) Pub. Date: Dec. 24, 2020

(54) PAPER-BASED COLLECTION AND TEST DEVICES FOR BIOLOGICAL SAMPLES

(71) Applicant: Ohio State Innovation Foundation, Columbus, OH (US)

(72) Inventors: Abraham K. BADU-TAWIAH, Columbus, OH (US); Deidre E. DAMON, Columbus, OH (US); Suji LEE, Columbus, OH (US); Benji FREY, Columbus, OH (US)

(21) Appl. No.: 16/979,295

(22) PCT Filed: Mar. 11, 2019
(86) PCT No.: PCT/US2019/021696

§ 371 Date: Sep. 9, 2020

Related U.S. Application Data
(60) Provisional application No. 62/640,628, filed on Mar. 9, 2018.

(51) Int. Cl. G01N 33/543; G01N 33/548

(52) U.S. Cl. CPC G01N 33/54306; G01N 33/54353; G01N 33/548

(57) ABSTRACT

Disclosed herein systems, apparatuses and methods for the collection and testing of biological samples, and more specifically to rapid and simple methods for diagnosis of various diseases, conditions, or symptoms via the testing of collected biological samples on paper devices.

### CROSS-REFERENCE TO RELATED APPLICATION

This application claims the benefit of U.S. Provisional Application 62/640,628, filed on Mar. 9, 2018, the contents of which is hereby incorporated in their entirety.

### FIELD OF THE INVENTION

The present invention relates generally to systems, apparatuses and methods for the collection and testing of biological samples, and more specifically to rapid and simple methods for diagnosis of various diseases, conditions, or symptoms via the testing of collected biological samples on paper devices.

### BACKGROUND

The collection of biological samples (such as biological fluid samples), and the use of those samples in diagnosing various diseases or conditions, is a long-lived practice. For more than a century, biological fluid samples (e.g., blood) have been collected onto paper as dried blood spots (DBS). The dried samples can easily be shipped to an analytical laboratory and analyzed using various methods such as DNA amplification or HPLC. More specifically, dried blood spot specimens are collected by applying a few drops of blood, that may be drawn by lancet from the finger, heel or toe, onto specially manufactured absorbent filter paper. The blood is allowed to saturate the paper and air dry (for 30 minutes up to several hours). Specimens may be stored in low gas-permeability plastic bags with desiccant added to reduce humidity, and may be kept at ambient temperature, even in tropical climates.

Once in the laboratory, the sample may be processed. For example, technicians may separate a small disc of saturated paper from the sheet using an automated or manual hole punch, dropping the disc into a flat-bottomed microtiter plate. The blood is eluted in phosphate buffered saline containing 0.05% Tween 80 and 0.005% sodium azide, overnight at 4° C. The resultant plate containing the eluates forms the “master” from which dilutions can be made for subsequent testing. Alternatively, automated solutions may extract the sample by flushing an eluent through the filter without punching it out.

Even though the basic DBS technology and method has been used for over a century, all noted advantages of DBS are still valid today—including the simplification of (small) sample collection, transportation, storage, and processing. Additionally, interest in the DBS method has been rekindled in recent years due to the emergence of personalized healthcare. This includes the recent introduction of an on-demand diagnostic strategy, which is expected to enable timely initiation of treatment and long-term disease monitoring within the context of a medical home and subspecialty center. These efforts are important, especially for newborn screening programs, the development of low-cost analytical diagnostic methods for use in resource-limited settings, environmental research, and drug analysis. In view of the increasing interest in the use of DBS technology, there are certain aspects of DBS that need improvement, including the preservation of labile compounds during storage, while maintaining the simplicity of the approach.

In that regard, the current basic precautions used during DBS collection include limiting sample exposure to moisture, sunlight, and heat (as those can harm and degrade a collected sample, thereby negatively affecting analyte integrity). However, simple exposure of DBS to ambient air can also substantially affect analyte integrity.

Additionally, because a current focus of DBS collection is re-testing at a reference laboratory (which may be a part of external equality assessment plan), it has become critical to know the exact volume of blood in the punched sample to enable effective comparison to results recorded at the testing site. This seemingly simple task is complicated by (1) volcanic effects—which cause a concentration gradient in DBS, with higher analyte concentrations detected toward the edge of the dried blood spot; (2) chromatographic effects—e.g., the choice of paper substrate impacts DBS sampling by altering blood diffusion and adsorption; and (3) hematocrit effects—varied red blood cells inpatients' blood (e.g., anemic sample) cause variable blood diffusion on paper, altering volume sampled in a punch. As such, the determination of blood volume in punched paper samples is currently achieved using mathematical calculations or via radioactive chemical tracers. However, these are not always accurate, due to the drawbacks listed above.

In view of (1) the problems with samples being susceptible to oxidative stress from atmospheric air that negatively affects the integrity of any analyte or analytes, (2) the difficulty in knowing the volume of blood being tested, and (3) other drawbacks, improvements in the current methods are needed, while maintaining the ease and simplicity of sample collection and testing.

Apart from the drawbacks present in current methods of collection of a test sample (whether via DBS or other method), there are drawbacks to current diagnostic tests run on collected samples, which seek to test for many diseases, conditions, symptoms, etc.—for example, malaria, colorectal cancer, and others.

In that regard, one issue that often presents itself is that many current blood tests for various diseases are not sensitive enough for accurate diagnosis in all cases. Examples of this can be seen in current diagnostic tests for malaria—such diagnostic methods including microscopy, rapid diagnostic tests, and polymerase chain reaction (PCR) Regarding microscopy: Changes in morphological appearances of Plasmodium parasite species, (due to drug pressure, strain variation, or approaches to blood collection) have created diagnostic problems that cannot easily be resolved merely through microscopic examination. Pregnant women (and to a lesser extent, children under 5 years old) are particularly at a disadvantage when their peripheral blood smear is used for malaria diagnosis because of the occurrence of pregnancy-associated malaria (PAM), the biology of which differs significantly from that seen in peripheral blood. PAM accounts for a third of the preventable low birth weight babies in sub-Saharan Africa, and close to 200,000 infant deaths annually. In PAM, sequestration and cytoadherence of parasitized erythrocytes reduces the number of circulating ring-stage parasites in the peripheral blood. Thus, visualization of parasites using microscopy is typically not suited for detecting malaria infection during pregnancy.

Rapid diagnostic test (RDT) methods based on antigen biomarkers are reported to have better sensitivity than microscopy in diagnosing PAM. However, a major constraint for RDT is the need to obtain blood samples, which can be problematic (i.e., requiring trained personnel to minimize attendant risk of infection) when collected from infants, young children, and pregnant women. In addition, the sensitivity of an RDT is not adequate to enable diagnosis using non-invasive samples such as saliva and urine.

Additionally, RDTs are not currently home-based. Although improving patients' involvement in their own diagnosis and treatment is increasingly being encouraged in every area of medicine, most studies that have evaluated the performance of rapid malaria diagnostic devices have been performed in a hospital setting. The ultimate goal should be to use the device at home, by a family member or local health care worker. Such an endeavor is difficult for RDT due to a host of reasons. A first reason is because of the generation of time-dependent results due to the use gold- or enzyme-conjugated antibodies—i.e., specific read-out time is required to ensure the validity of test. Failure to follow such simple instructions (e.g., when to read test results) however are primarily responsible for false positive results in RDTs. Second, the RDT device is not stable enough under conditions commonly found in malaria endemic regions—discoloration of negative controls is the main damage to the device making it difficult to discriminate against positive test results. And third, the test produces inadequate accuracy and inconsistent results compared to a health-outpost utilizing centralized detection. In view of the above drawbacks with current such diagnostic tests, it is necessary to develop surveillance strategies that seek to empower the at-risk population to manage their own health.

PCR, due to its high sensitivity, is currently proposed as the method of choice for non-invasive analysis for malaria diagnosis. PCR, however, requires a multitude of sample preparation steps and precise reagent temperature control to reach the desired analyte state that can be handled by the instrument. As a consequence, large sample volumes (0.2-1 mL) are needed with concomitant increase in analysis time.

In view of all of these (and other) drawbacks in current methods for detecting malaria (and other diseases which may use the same or similar diagnostic methods), an improved method for detecting malaria, or other diseases that are commonly detected using above-described methods, is needed.

Another example of a disease which suffers drawbacks in its methods for sample collection and testing for diagnosis is colorectal cancer (CRC). CRC is the third most common cancer in westernized countries. Over 50,000 people die each year from CRC in the United States alone. Many countries, including the U.S., have Bowel Cancer Screening Programs (BCSP) which aim to detect polyps and early cancers, resulting in pressure on endoscopy services. In countries like Japan, the United Kingdom, and Australia, CRC screening is based on fecal occult blood (FOB) in fecal smears. Simulation models have demonstrated that a screening program centered on FOB achieves 94% of the benefit that an all-colonoscopy program is able to accomplish, but at a lower cost per life year gained. This is attractive, but until recently, FOB has used guaiac testing. However, not all early neoplasia bleed and other pathology may bleed. Thus, FOB testing is susceptible to false positive and negative outcomes.

The United States Preventive Services Task Force (USPSTF) recommends screening for CRC beginning at age 50 and continuing until age 75. The current patient self-test encourages the individual to collect samples of their own stool over a consecutive, three-day period. However, there are drawbacks to the current test methods. For example, while the actual process of sampling doesn't take long, it can be unpleasant and embarrassing for the individual. The sample must also be collected without getting wet which may cause difficulty for some, particularly older patients. These factors tend to lead to a low compliance rate. Furthermore, the test itself has poor sensitivity for early stage detection, which means that many patients go undiagnosed until the disease is at a more advanced stage leading to poor survival outcomes. Thus, an improved method for detecting colorectal cancer, or other diseases that are commonly detected using above described methods (such as FOB testing on self-collected samples), is needed.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention is directed to a paper-based blood collection platform that collects and uses three-dimensional dried spheroids as opposed to the traditional two-dimensional DBS sample collection procedure. In one embodiment, this collection procedure uses functionalized hydrophobic paper substrates to overcome the challenges associated with the traditional DBS procedure. As will be discussed in greater detail below, a biological sample applied on the hydrophobic paper forms a spherical drop due to a mismatch in surface energies, which dries to yield a dried spheroid.

Via the use of 3D spheroids (instead of the traditional 2D DBS collection), hydrolytically labile compounds such as cocaine and diazepam trapped in the 3D dried spheroids are stabilized, compared with storage done under the porous DBS conditions where a major portion of the sample becomes susceptible to oxidative stress from atmospheric air. Additionally, because the origin of volcanic, chromatographic, and/or hematocrit effects can all be traced to a common source—uneven biofluid/analyte adsorption—controlling wetting on hydrophobic paper provides easy validation of results without the use of chemical tracers to estimate sample volume in dried punch. Further, the hydrophobic paper strips of this aspect of the present invention also provide the ability for direct mass spectrometry (MS) detection through paper spray (PS) ionization for sensitive analyte quantification. In-situ extraction of illicit drugs (e.g., cocaine, benzoylecgonine, amphetamine, and/or methamphetamine) from the dried blood spheroids results in sub-ng/mL limit of detections. And further still, proper control of the analyte desorption from the paper substrate provides a new electrostatic spray-based method to estimate the surface energies of the hydrophobic paper strips, which is more effective than the conventional approach based on contact angle measurements.

Another aspect of the present invention provides a simple test apparatus and method that allows an individual to perform a finger prick blood sample on to a paper-based assay for early disease detection in a manner that will be faster, simpler, and cheaper than those currently available and will be more sensitive for early stage detection, less susceptible to false positive/negative outcomes, and technologically flexible allowing the process to be readily refined as new biomarkers become viable.

In a specific embodiment of this aspect of the present invention—centering on testing for CRC—a BCSP may be provided whereby an individual receives a test-kit in the mail. The individual uses a finger prick stick to collect a blood sample on a paper substrate. This takes a matter of seconds, is simple to convey, and easy to perform. The blood is dried in ambient conditions over a matter of hours. The sample is then be placed in a pre-addressed envelope and posted to a centralized lab. In the laboratory, the paper substrate would be analyzed through an on-chip paper electrospray mass spectrometry technique to yield a quantitative determination of the given biomarkers.

One aspect of the present invention provides improved collection, stabilization, and detection of protein biomarkers, without the need for cold storage. In that regard, an antibody-bound paper is used for sample collection; and labile protein biomarkers are selectively captured immediately upon sample application onto the paper device. Detection of the captured protein may be achieved (in one embodiment) through a sandwiched immunoassay with a reporter antibody that is also specific to the protein biomarker of interest. A reporter compound can be generated from the reporter antibody, and detected using mass spectrometry. Due to the high sensitivity of mass spectrometry for small molecules, sandwich complexes can be detected at low as picomolar concentrations. Unlike enzymes or gold nanoparticles, the immunoassay products (a “sandwich complex”) are stable, permitting easy storage and transport of the paper device. Therefore, immunoassays performed as described are highly stable and able to be stored prior to analysis for extended periods of time.

Also disclosed are three-dimensional platforms for the analysis of biological samples. The platforms include multiple layers including capture and reporter antibodies. Using conventional printing techniques, the platform can include multiple zones, each containing a different capture/reporter antibody system. The generated reporter compounds can be combined and detected at the same time using mass spectrometry.

The details of one or more embodiments are set forth in the descriptions below. Other features, objects, and advantages will be apparent from the description and from the claims.

### BRIEF DESCRIPTION OF THE FIGURES

FIG. 1 is an illustration of cell exosomes represented by small vesicles of different sizes that are released by fusion of multivesicular endosomes with the plasma membrane.

FIG. 2 illustrates a typical geographical location for city (C), town (T), and village (V) in a Ghanaian community.

FIG. 3 is a schematic representation of an experimental set-up of paper spray ionization. The black triangle is a 2D wax-printed paper for paper spray.

FIG. 4 is a schematic representation of an experimental setup using (A) paper triangles and (B) paper rectangles. Image (C) shows a 4 μL dried blood spot/spheroid on an untreated (left) and treated (right) paper substrates, including the front (top) and back (bottom). (D) shows workflow of direct on-surface dried blood analysis.

FIG. 5 shows spectra grouped by paper treatments (Columns), and spectra grouped by drugs fragmented (Rows). Panels A-D show fragmentation of amphetamine (A), methamphetamine (B), benzoylecgonine (C), and cocaine (D) on untreated paper strips. Panels E-H show fragmentation of amphetamine (E), methamphetamine (F), benzoylecgonine (G), and cocaine (H) on 2 hour treated paper strips. Characteristic fragments include: cocaine (304→182), benzoylecgonine (290→168), methamphetamine (150→119) and amphetamine (136→119).

FIG. 6 shows spectra grouped by paper treatments (Columns), and shows spectra grouped by drugs fragmented (Rows). Panels A-D show fragmentation of amphetamine (A), methamphetamine (B), benzoylecgonine (C), and cocaine (D) on paper strips treated for 30 minutes. Panels E-H show fragmentation of amphetamine (E), methamphetamine (F), benzoylecgonine (G), and cocaine (H) on paper strips treated for 2 hours. Characteristic fragments include: cocaine (304-182), benzoylecgonine (290→168), methamphetamine (150→119) and amphetamine (136→119).

FIG. 7 are photographs with panel A showing setup of a paper strip in front of the mass spectrometer with a dried blood spot immobilized on the surface. Panels B and C are images from a Watec camera showing a closer look at the loose paper fibers on the edge of the paper strip. (B) is a side on view and (C) is a top-down view. Fibers measure to be approximately 0.04 mm in diameter.

FIG. 8 is a graph showing total ion chromatogram of paper strip with alternating 3 kV and 0 kV applied. Sample is a dried blood spot on 2 hour treated paper with 20 μL ethyl acetate applied.

FIG. 9 is a graph with panel A showing stability of cocaine in dried blood, panel B showing neat dried diazepam prepared in water, and panel C showing diazepam in dried-14-blood. Both dried blood spots (untreated) and spheroids (treated) samples were stored under ambient conditions at 25° C. Internal standard was spiked into the spray solvent to normalize between samples and days.

FIG. 10 are graphs showing offline analysis of 2 μg/mL cocaine in dried bloodspot on untreated paper and dried blood spheroid on 30 minute and 2 hour treated paper. Sample was spotted and stored for 1 and 2 days at 25° C. The samples were then extracted in ethyl acetate for 30 minutes in a sonicator. Extract was then nanosprayed, and m/z 304 (cocaine) and 290 (benzoylecgonine, possible cocaine degradation product) were fragmented.

FIG. 11 is a graph showing stability of benzoylecgonine in dried blood spots (untreated) and spheroids (treated) stored in ambient conditions at 25° C. Internal standard was spiked into the spray solvent to normalize between samples and days.

FIG. 12 is a graph showing stability of 2 μg/mL cocaine and benzoylecgonine in dried blood spots/spheroids on untreated, 30-minute treated paper, and 2 hour treated paper. Samples were stored in a desiccator for 15 days and then analyzed with 5 kV and 10 μL ethyl acetate containing 500 ng/mL deuterated internal standard to normalize between samples and between days.

FIG. 13 is a graph showing CID of neat diazepam, m/z 285 in panel A; CID of 02 adduct of diazepam (m/z 317) in water immediately after depositing on a paper triangle in panel B; and CID of O₂ adduct of diazepam in water 4 days after depositing on a paper triangle in panel C.

FIG. 14 is a graph showing the contact angle of DI water deposited onto filter paper with varying treatment times of vapor phase silane.

FIG. 15 is a graph showing, in panel A, observation of ion intensity varying with the change of surface tension of ACN/H2O spray solvents (Table 2). Peak surface tensions are used as values for y-axis in plot B. Calibration of cellulose acetate and polycarbonate, with treated and untreated paper projected onto the line is shown in panel B. The determined surface energies of paper substrates are provided in Table 3.

FIG. 16 is a graph showing heat transient simulation analysis. Both blood storage geometries had an initial temperature of 30° C. and were subject to a constant ambient air temperature of 40° C. Temperature is measured at the geometric center for each case.

FIG. 17 is a graph showing data found on FIG. 15, panel A, fitted with Equation S2. Fitting parameters were found to be: a=106 m2/mN2, b=66.5 mN/M, c=2885 mN2/m2.

FIG. 18 is a graph showing a Mathematica plot of Equation 1 using parameters found for fitting in FIG. 17.

FIG. 19 are photographs showing acetonitrile/water droplets of varying ratios (see Table 2, below) resting on a paper strip treated for 2 hours when 5 kV is applied. In panel A, droplet includes solvent 7 (pure acetonitrile, surface tension 29 mN/m). In panel B, droplet includes solvent 5 (surface tension 38 mN/m). In panel C, droplet includes solvent (surface tension 41 mN/m). In panel D, droplet includes solvent 1 (surface tension 62 mN/m).

FIG. 20 are photographs of (A) front and (B) back of untreated and treated paper with 4 μL whole blood dried for 24 hours. Time listed is the amount of time gas phase silane is allowed to react with the paper surface.

FIG. 21 are graphs showing extraction from dried blood spots with 20 μL ethyl acetate. Absolute intensity of 500 ng/mL amphetamine, methamphetamine, cocaine, and benzoylecgonine on the surface of paper triangles. Characteristic fragments of cocaine (304→182), benzoylecgonine (290→168), methamphetamine (150→119) and amphetamine (136→119) were used for quantification. These triangles were untreated and treated for 5, 30, 120, 240, 720, and 1440 minutes with silane.

FIG. 22 are graphs showing optimization of treatment time of paper using common illicit drugs and extraction from dried blood spots with 20 μL acetonitrile. Absolute intensity of 500 ng/mL amphetamine, methamphetamine, cocaine, and benzoylecgonine on the surface of paper triangles. Quantification of characteristic fragments of cocaine (304→182), benzoylecgonine (290→168), methamphetamine (150→119) and amphetamine (136→119) was performed. These triangles were untreated and treated for 5, 30, 120, 240, 720, and 1440 minutes with silane.

FIG. 23 are graphs with A, C, and E showing calibration of cocaine ranging from 10-500 ng/mL in dried blood spots, and B, D, and F representative mass spectra of fragmentation of cocaine with a concentration of 10 ng/mL on untreated paper (A and B), paper treated for 30 minutes (C and D), and paper treated for 2 hours (E and F). Mass spectra show the increased signal to noise of cocaine on paper treated for 30 minutes when compared to the untreated and 2 hour treated paper, which was expected, as shown by the optimization in FIG. 21. Error bars show one standard deviation of trials performed in triplicate.

FIG. 24 shows graphs with A, C, and E showing calibrations of benzoylecgonine in dried blood on (A) untreated paper triangles, (C) 30-minute treated paper triangles, and (E) 2 hour treated paper triangles. Error bars are one standard deviation. B, D, and F are sample MS/MS spectra from the respective paper treatments at 10 ng/mL concentration of benzoylecgonine.

FIG. 25 shows graphs of calibrations of methamphetamine in dried blood on (A) untreated paper triangles, (B) 30-minute treated paper triangles, and (C) 2 hour treated paper triangles. Error bars are one standard deviation.

FIG. 26 shows graphs with A, C, and E showing calibrations of amphetamine in dried blood on (A) untreated paper triangles, (C) 30-minute treated triangles, and (E) 2 hour treated paper triangles. Error bars are one standard deviation. B, D, and F show sample MS/MS spectra from respective paper treatments at 10 ng/mL concentration of amphetamine.

FIG. 27 illustrates a proposed synthetic reaction scheme for a pH-active ionic probe.

FIG. 28 illustrates the ESI-MS spectrum of purified reaction products (A) ITEA and (B) ITBA. Inserts were recorded after solution-phase hydrolysis/cleavage of reaction product. (C) Hydrolytic kinetics for ITEA at different pHs. (D) Stability of ITEA and ITBA was investigated where percent of intact probes remained constant over 30 days at pH 7.

FIG. 29 are graphs showing ESI-MS characterization of (A) pure antibody and (B) ionic probe modified antibody. Charge state is 52+. The peaks labelled with red numbers come from the conjugated antibodies.

FIG. 30 illustrates other means of stimulating the selected ionic probes including (A) UV-light illumination, and (B) redox chemistry.

FIG. 31 illustrates synthesis of colloidal gold for mass spectrometry signal amplification. Large quantities of the ionic probe will be caused to self-assemble at the gold surface by using excess amount of product 5 over 6, as illustrated by the insert. Photograph in insert shows three different sizes (15 nm, 25 nm, and 40 nm) of gold nanoparticles.

FIG. 32 is a schematic representation showing MS signal amplification through amine oxidation using on-surface photo-redox reactions with portable laser pointer.

FIG. 33 is a schematic representation of the capture of analyte between two monoclonal antibodies followed by the release of ionic species for detection by MS.

FIG. 34 illustrates the analysis of a PfHRP-2 malaria antigen from serum samples using the paper-based immunoassay utilizing ITBA ionic probe as mass reporters: (A) entire PfHRP-2 concentration range tested, (B) linear concentration range yielding LOD of 1.5 fmole per test zone, (C) Stability of the ionic probe in immunoassay demonstrated in MS analysis of positive (PfHRP2, 10 nM) and negative control test zones stored before the hydrolysis reaction. Signal from analyte was compared with that of an internal standard (A/IS); and (D) optical density (O.D.) value for ELISA assay of PfHRP2 (2.7 nM) after storage under Tris buffer solution (black) or dry (red) conditions before the addition of substrate. Each datum point is an average of eight replicates and error bars indicate standard deviation.

FIG. 35 is a schematic representation showing the proposed mechanism for the photo-catalyzed oxidation of triethanolamine (TEA) to both the aldehyde product (top) and hydrolysis product, diethanolamine (DEA) (bottom).

FIG. 36 is a graph of a real-time photo-reaction screening mass spectrum for a 1 ppm solution of triethanolamine containing 25 μM Eosin Y. This spectrum was collected at the onset (time=0 minutes) laser illumination.

FIG. 37 is a graph showing a real-time photo-reaction screening mass spectrum for a 1 ppm solution of triethanolamine containing 25 μM Eosin Y. This spectrum was collected after 1.28 minutes laser illumination time.

FIG. 38 is a graph showing a real-time photo-reaction screening mass spectrum for a 1 ppm solution of triethanolamine containing 25 μM Eosin Y. This spectrum was collected after 1.86 minutes laser illumination time.

FIG. 39 illustrates the capture of analyte between two monoclonal antibodies followed by on-demand MS analysis through amine oxidation using on-surface photo-redox reactions with portable laser pointer.

FIG. 40 are graphs showing stability of the ionic probe and enzyme involved in immunoassay. a) MS analysis results of positive (PfHRP2, 10 nM) and negative control test zones stored before probe cleavage, b) Optical density (O.D.) values of ELISA assay of PfHRP2 (2.7 nM) after storage under Tris buffer solution (black) or dry (red) conditions before the addition of substrate.

FIG. 41 is a deconstructed perspective view of a prototype 3D paper device for multiplexed malaria detection. The device is capable of (1) precise measurement of biofluid volume (topmost layer), (2) on-surface sample splitting, (3) hydrophobic layer to control reaction time, and (4) on-chip MS.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

The term “alkyl” as used herein is a branched or unbranched saturated hydrocarbon group of 1 to 24 carbon atoms, such as methyl, ethyl, w-propyl, isopropyl, n-butyl, isobutyl, s-butyl, t-butyl, w-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can include one or more elements of unsaturation, e.g., alkene and/or alkyne. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

As used herein, “alkyl” is generally used to refer to both unsubstituted alkyl groups and substituted alkyl groups; however, substituted alkyl groups are also specifically referred to herein by identifying the specific substituent(s) on the alkyl group.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As described above, there are numerous drawbacks to current methods of collecting biological samples, and numerous drawbacks to current methods of testing collected biological samples for the diagnosis of various conditions. The various aspects of the present invention serve to reduce and/or eliminate the drawbacks discussed.

Disclosed herein are apparatuses, systems and methods for analyzing biological samples. In some embodiments, the biological sample to be analyzed include extracellular fluid (i.e., fluid occurring outside of cells), intracellular fluid (i.e., fluid occurring within cells), transcellular fluid (fluids formed from transport activity in cells), and biological tissues. In some embodiments, the analyte can include urine, whole blood, blood serum, plasma, lymph, saliva, sweat, tears, cerebrospinal fluid, ocular fluid, joint fluid, gastrointestinal fluid, stomach acid, pancreatic fluid, serous fluid, synovial fluid, aqueous humor of the eye, perilymph, or endolymph.

In certain embodiments, the apparatuses, systems and methods can be used to detect and quantitate small molecule compounds in the biological sample, including illicit drugs and performance enhancing compounds, as well as their metabolites. In some cases, the apparatuses, systems and methods can be used to detect and quantitate antigens, for instance those indicating a particular medical condition or disease state.

One aspect of the present invention is directed to a paper-based collection platform that forms and uses three-dimensional dried spheroids as opposed to the traditional two-dimensional sample collection procedure. In one embodiment, this new dried sample collection procedure uses functionalized hydrophobic paper substrates to overcome the challenges associated with the traditional procedure. As will be discussed in greater detail below, a sample applied on the hydrophobic paper forms a spherical drop due to a mismatch in surface energies, which dries to yield a dried spheroid. In preferred embodiments, the biological sample is blood, either whole blood, blood serum, or blood plasma.

As used herein, a paper substrate includes a cellulosic component. Exemplary cellulosic materials include cotton, kenaf, flax, hemp, jute, rayon, sisal, caroa, banana, coconut, wool, rye, wheat, rice, sugar cane, bamboo, or a combination thereof. In some instances, the substrate can also include synthetic materials, for instance carbon fibers, polyethylenes, polyesters, polyamides, phenol-formaldehydes, polyvinyl chlorides, polyurethanes, or a combination thereof. When the substrate is a mixture of cellulosic and synthetic materials, it is preferred that the cellulosic material constitutes at least 50%, at least 60%, at least 70%, at least 80%, or at least 90% of the total substrate weight.

Cellulosic substrates suitable for the disclosed systems, methods, and apparatuses can be from about 5-100 wt., from about 5-80 wt., from about 10-70 wt., from about 20-60 wt., from about 30-50 wt., from about 10-50 wt., from about 10-30 wt., from about 20-40 wt., from about 10-20 wt., from about 40-60 wt., or from about 60-80 wt.

In certain embodiments, the paper substrate may be functionalized. The hydroxyl functional groups present in cellulosic materials may be capped with hydrophilic or hydrophobic groups. Exemplary functional groups include silanes, which may be installed by reacting paper substrate with a compound having the formula:

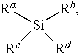

wherein R^{a}, R^{b}, R^{c}, and R^{d} are independently selected from OH, R^{x}, OR^{x}, NHR^{x}, N(R^{x})₂, OC(O)R^{x}, F, Cl, Br, or I, wherein R^{x} is in each case selected from C₁₋₁₂alkyl, aryl, heteroaryl, and heterocyclyl, and wherein any two or more of R^{a}, R^{b}, R^{c}, and R^{d} can together form a ring. Suitable silanes may be installed by contacting paper with a vapor that includes the silane compound.

In some embodiments, the paper substrate can have a thickness from 50-1,000 μm, from 50-900 μm, from 50-800 μm, from 50-700 μm, from 50-600 μm, from 50-500 μm, from 50-400 μm, from 50-300 μm, from 50-200 μm, from 100-300 μm, from 150-350 μm, or from 150-250 μm.

Paper substrate suitable for use in the disclosed invention may be characterized by their surface energy. For instance, in some embodiments, the paper substrate can have a surface energy no greater than 30 mN/m, no greater than 32.5 mN/m, no greater than 35 mN/m, no greater than 37.5 mN/m, no greater than 40 mN/m, no greater than 42.5 mN/m, no greater than 45 mN/m, no greater than 47.5 mN/m, or no greater than 50 mN/m. The surface energy of the paper substrate can be determined using surface energy estimation via electrostatic spray described below.

In certain aspects, the paper substrate is shaped to include at least one tip, for instance, the shape of a triangle, including equilateral, isosceles, and scalene triangles. The tip serves to direct the ionized compounds toward the inlet of the mass spectrometer or other detector. Although any type of triangle may be employed, in some embodiments it is preferred that the paper substrate is an isosceles triangle. For isosceles substrates, the apex angle can be from 5°-45°, from 10°-40°, from 15°-40°, from 20°-40°, from 25°-40°, from 30°-40°, 5°-35°, from 10°-35°, from 15°-35°, from 20°-35°, from 25°-35°, from 30°-35°, from 5°-25°, from 10°-25°, from 15°-25°, or from 20°-25°. In some embodiments, the height (i.e., the length to perpendicular bisector to the base) can be at least 150% the length of the base, at least 175% the length of the base, at least 200% the length of the base, at least 225% the length of the base, at least 250% the length of the base, at least 275% the length of the base, or at least 300% the length of the base. In certain embodiments, the height of the triangle can be from 100-300% the length of the base, from 100-250% the length of the base, from 100-200% the length of the base, from 100-150% the length of the base, from 150-300% the length of the base, from 150-250% the length of the base, or from 200-250% the length of the base.

In certain aspects, a portion of the paper can include a hydrophobic material to further direct the biological sample to the tip. For instance, and as shown in FIG. 3, the paper substrate can be infused with a hydrophobic material defining a reservoir in fluid communication with a channel leading to the tip. In certain embodiments, the channel is disposed along the perpendicular bisector of the triangle. Suitable non-absorptive materials include waxes, polyethylenes, polypropylenes, polyacrylates, polystyrenes, rubbers, polystyrenes, and copolymers thereof. The hydrophobic portions can be installed using photolithography, inkjet etching, inkjet printing, ink stamping, plasma treatment, laser treatment, screen printing, or lacquer spraying.

When a biological sample is contacted with the paper substrate, the mismatched surface energy between the liquid and the paper causes the biological sample to “bead up,” or take the shape of a sphere. Such materials are designated herein as “3D spheroids.” Spheroids may be oblate spheroids, prolate spheroids, or sphere shaped. The spheroid may or may not be partially absorbed into the paper substrate. However, it is preferred that the height of the spheroid (i.e., the distance between the surface of the paper substrate and the highest point on the spheroid,) is at least 25% the diameter (i.e., taken in the directed parallel to the surface of the paper substrate. In some embodiments, the height is at least 30% the diameter, at least 35% the diameter, at least 40% the diameter, at least 45% the diameter, at least 50% the diameter, at least 55% the diameter, at least 60% the diameter, at least 65% the diameter, at least 70% the diameter, at least 75% the diameter, or at least 80% the diameter.

In some embodiments, a viscosity modifier can be added to the biological sample in order to enhance its propensity to form a spheroid upon contact with the paper substrate. Exemplary viscosity modifiers include xanthum gum, polyvinylpyrrolidinone, polyethylene glycol, hydroxypropyl cellulose, maltodextrin, sodium starch glycolate, and others. Viscosity modifiers are especially useful with less viscous biological fluids such as urine. The viscosity modifier may be present in an amount from 0.5-100 wt % relative to the total mass of the sample. In some embodiments, the viscosity modifier can be present in an amount from 0.5-15 wt %, from 0.5-10 wt %, from 0.5-5 wt %, from 0.5-2.5 wt %, from 5-15 wt %, from 10-20 wt %, from 15-25 wt %, from 20-30 wt %, from 25-35 wt %, from 30-40 wt %, from 35-45 wt %, from 40-50 wt %, from 45-55 wt %, from 50-60 wt %, from 55-65 wt %, from 60-70 wt %, from 65-75 wt %, or from 60-100 wt %.

Via the use of 3D dried spheroids (instead of the traditional 2D collection), hydrolytically labile chemicals, for instance cocaine and diazepam, are stabilized compared with storage done under the porous conditions where a major portion of the sample becomes susceptible to oxidative stress from atmospheric air. Additionally, because the origin of volcanic, chromatographic, and/or hematocrit effects can all be traced to a common source—uneven biofluid/analyte adsorption—controlling wetting on hydrophobic paper enables easy validation of results without the of use chemical tracers to estimate sample volume in the dried punch. Further, the hydrophobic paper strips provide the ability for direct mass spectrometry (MS) detection through paper spray (PS) ionization for sensitive analyte quantification, an ambient ionization technique that allows MS analysis of analytes present on an ordinary paper surface cut to a sharp tip (FIG. 3). Unlike other ambient ionization techniques (e.g., desorption electrospray ionization), PS is well suited for on-site in situ analysis as pneumatic assistance is not needed to transport the analyte to the inlet of the mass spectrometer. Transfer of analytes occurs when the sample present on the paper triangle is solubilized by applying a spray solvent. Under this condition, charged micro-droplets are emitted from the tip of the wet paper triangle after applying 3-5 kV DC voltage to the paper triangle.

Exemplary spray solvents include organic solvents, water, and combinations thereof. Suitable organic solvents include methanol, ethanol, isopropanol, methylene chloride, chloroform, diethyl ether, tetrahydrofuran, acetonitrile, acetone, ethyl acetate, methyl ethyl ketone, hexanes, toluene, and others. The skilled person can select an appropriate solvent to solubilize the target analytes in the spheroid.

Also provided herein are diagnostic kits. The kits may be used in an in-home, in-patient or in-office setting. In some embodiments, an individual can receive a test-kit in the mail. In addition to the paper substrates described herein, the kit will also include one or more finger prick sticks. The individual uses a finger prick stick to collect a blood sample on the disclosed paper substrates. This takes a matter of seconds, is simple to convey, and easy to perform. The blood can then dry in ambient conditions over a matter of hours. The sample can be placed in a pre-addressed envelope and posted to a centralized lab. In the laboratory, the paper substrate would be analyzed through an on-chip paper electrospray mass spectrometry technique to yield a quantitative determination of the given biomarkers.

In certain embodiments, the paper substrates can be deployed to detect the presence of an antigen using reporter antibodies. Although mass spectrometer analysis of intact proteins and antibodies is possible, large and expensive devices are still required. As used herein, a reporter antibody is capable of generating and/or releasing a reporter compound (i.e., less than 300 Da) that is easily detected using small-footprint, low cost instruments, such as portable mass spectrometers. The reporter compounds are analyzed with high sensitivity and specificity. In essence, instead of directly analyzing a high-molecular weight protein, which generally requires a large, high-resolution MS instrument, the present invention permits the detection of a small molecule of defined mass, which can be readily accomplished on any mass spectrometer with atmospheric pressure interface (including portable instruments).

Because the sandwich immunoassay is analyzed using small molecule mass spectrometry, antigens can be detected at extremely low concentrations, for instance nanomolar, picomolar, or femtomolar concentrations. Because antigens are present immediately following infection, the systems disclosed herein can be used to diagnose infection at an extremely early stage.

The system includes a paper substrate conjugated to a capture antibody. The capture antibody binds the target antigen. The capture antibody may be conjugated to the paper substrate using conventional chemistries. In some embodiments, the capture antibody may simply be physically absorbed into the porous structure of the cellulose network. In other embodiments, a portion of the cellulose fibers may be modified to covalently conjugate with the capture antibody. In some embodiments, a portion of the cellulose fibers may be oxidized, e.g., to contain aldehyde groups, which then react with pendant amines in the capture antibody, resulting in a Schiff base, optionally using reductive conditions, resulting in a secondary amine. In certain embodiments, the cellulose can be reacted with a compound having a first functional group that forms a covalent bond with the primary hydroxyl groups in the cellulose (or an oxidized derivative thereof, e.g., aldehyde or carboxylic acid), and a second functional group that can covalently bind to the capture antibody, or the second functional group can be converted to a moiety that can bind to the capture antibody. Exemplary first functional groups include epoxides and primary amines, exemplary second functional groups include primary alcohols. As used herein, a paper substrate modified in this manner is said to have a spacer between the cellulose and capture antibody. In other embodiments, the paper substrate can be conjugated to avidin using the techniques described above, and combined with a biotin labeled capture antibody.

Subsequent to installation of the capture antibody, the system can be reacted with a blocking group, for instance tris(hydroxymethyl)aminomethane (“Tris”) in order to prevent non specific binding to the cellulose substrate.

The capture antibody-functionalized substrate is then contacted with a biological sample suspected of containing the antigen. The system is then contacted with a reporter antibody, resulting in a sandwich complex if antigen was present in the biological sample. After the sandwich complex has been formed, the system is washed to remove any unbound reporter antibody, and subsequently treated to generate a reporter compound. The presence of the reporter compound can be determined using mass spectrometry. In that regard, a capture-antibody-bound paper is used for sample collection; and antigens are selectively captured immediately when a biological fluid is contacted with the paper. Unlike enzymes or gold nanoparticles, the sandwich complexes are stable, permitting easy storage and transport of the paper device. While metal tags have been used to enable amplification of MS signals, their release and ionization requires plasma sources, which in turn requires pressurized gases such as helium. As such, in preferred embodiments of the invention, the reporter antibodies do not include exogenous metal tags.

In some instances, the paper substrate can be conjugated to a plurality of capture antibodies, permitting the detection of a plurality of target analytes. Provided that different reporter compounds are associated with different reporter antibodies, a plurality of different antigens can be identified in a single assay.

Exemplary antigens that may be detected include cancer antigens (including tumor antigens), viral antigens, bacterial antigens, fungal antigens, parasitic antigens, neuronal antigens, and others. In certain preferred embodiments, the antigen is a marker for HW, malaria, dengue, Chagas' disease, Leishmania, Trypanosoma, Plasmodium, Toxoplasma, adenovirus, Campylobacter, rotovirus, norovirus, E. coli, Salmonella, influenza, anthrax, Legionella, chlamydia, trachomatis, herpes simplex, gonorrhoeae, hepatitis (including A, B, C and other strains), measles, penuomonia, or tuberculosis.

The reporter antibody is functionalized to generate a small molecule reporter compound subsequent to sandwich complex formation. In some cases, the reporter antibody includes a quaternary ammonium group:

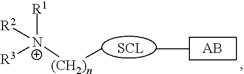

wherein AB is an antibody, SCL is a selectively cleavable linker, n is a number from 0-30 (e.g., 1-5, 2-7, 5-10, 5-15, 10-20, or 10-30), and each of R′, R², and R³ are independently selected from C₁₋₁₂alkyl, aryl, heteroaryl, and heterocyclyl, and wherein any two or more of R¹, R², and R³ can together form a ring. In a preferred embodiment, each of R¹, R², and R³ are methyl. For embodiments in which a plurality of capture antibodies are present, to form sandwich complexes with a plurality of reporter antibodies, it is preferred that the selectively cleavable linker is the same, but each reporter antibody includes a distinct constellation of R², and R³ groups, so that each reporter compound can be detected in the same mass spectrometer analysis.

Cleavage of the linker generates a free quaternary ammonium compound, which can be detected at very low concentration using mass spectrometry. The selectively cleavable linker may be cleaved in response to a pH change, irradiation, oxidant, or reductant. Exemplary pH sensitive linkers include esters (for cleavage by hydrolysis), exemplary oxidant cleaved linkers include diazos, exemplary reductant cleaved linkers include disulfides, and exemplary irradiation cleaved linkers include ortho-nitrobenzyl ethers. In some instances, the reporter antibody can include:

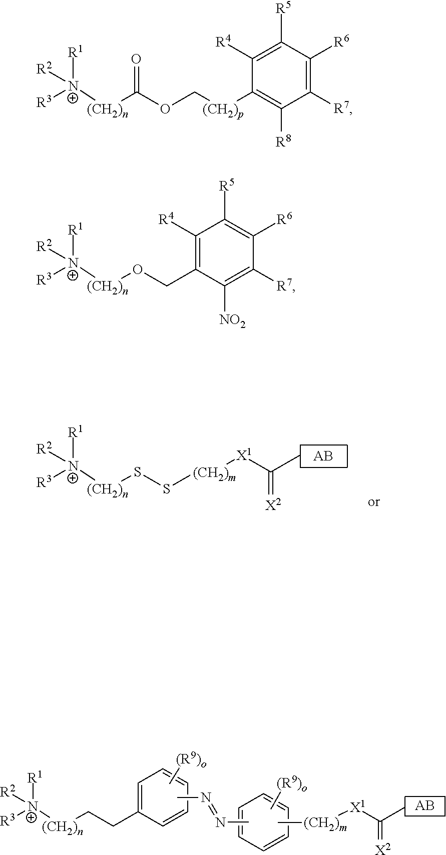

wherein AB, n, R¹, R², and R³ are as defined above;

X¹ is null, NH, O, or S, and X² is S or O;

m is a number from 0-20, 0-10, 0-5, 0-2, 2-20, 2-10, 2-5, 5-20, 5-10, or 10-20;

n is a number from 0-20, 0-10, 0-5, 0-2, 2-20, 2-10, 2-5, 5-20, 5-10, or 10-20;

p is a number from 0-20, 0-10, 0-5, 0-2, 2-20, 2-10, 2-5, 5-20, 5-10, or 10-20;

o is in each case independently selected from 0, 1, 2, 3, or 4;

wherein one of R⁴, R⁵, R⁶, R⁷, R⁸ (if present) is selected from:

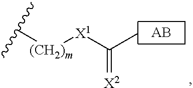

and the remaining groups are independently selected from OH, R^{a}, OR^{a}, NHR^{a}, N(R^{a})₂, C(O)R^{a}, OC(O)OR^{a}, OC(O)R^{a}, NO₂, cyano, F, Cl, Br, or I, wherein R^{a} is in each case independently selected from C₁₋₁₂alkyl, aryl, heteroaryl, and heterocyclyl; and

wherein R⁹ is in each case independently selected from OH, R^{a}, OR^{a}, NHR^{a}, N(R^{a})₂, C(O)R^{a}, OC(O)OR^{a}, OC(O)R^{a}, NO₂, cyano, F, Cl, Br, or I, wherein R^{a} is in each case independently selected from C₁₋₁₂alkyl, aryl, heteroaryl, and heterocyclyl. In certain preferred embodiments, R⁵, is alkoxy, e.g., methoxy, and R⁴ and R⁷ are each hydrogen.

In some embodiments, the selectively cleavable linker precursor compound includes an aldehyde:

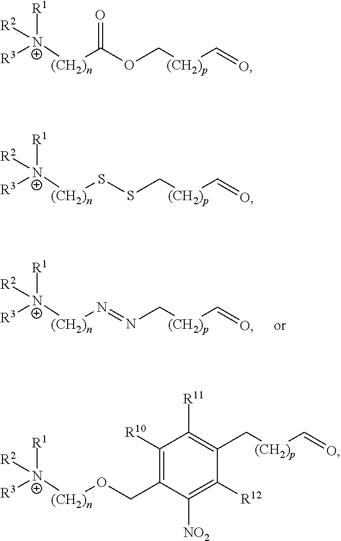

wherein n, p, R¹, R², and R³ are as defined above; R¹⁰, R¹¹ and R¹² are independently selected from OH, R^{a}, OR^{a}, NHR^{a}, N(R^{a})₂, C(O)R^{a}, OC(O)OR^{a}, OC(O)R^{a}, NO₂, cyano, F, Cl, Br, or I, wherein R^{a} is in each case independently selected from C₁₋₁₂alkyl, aryl, heteroaryl, and heterocyclyl. In certain preferred embodiments, R¹¹, is alkoxy, e.g., methoxy, and R¹⁰ and R¹² are each hydrogen. The precursor compound can be reacted with pendant amines in the reporter antibody as described above

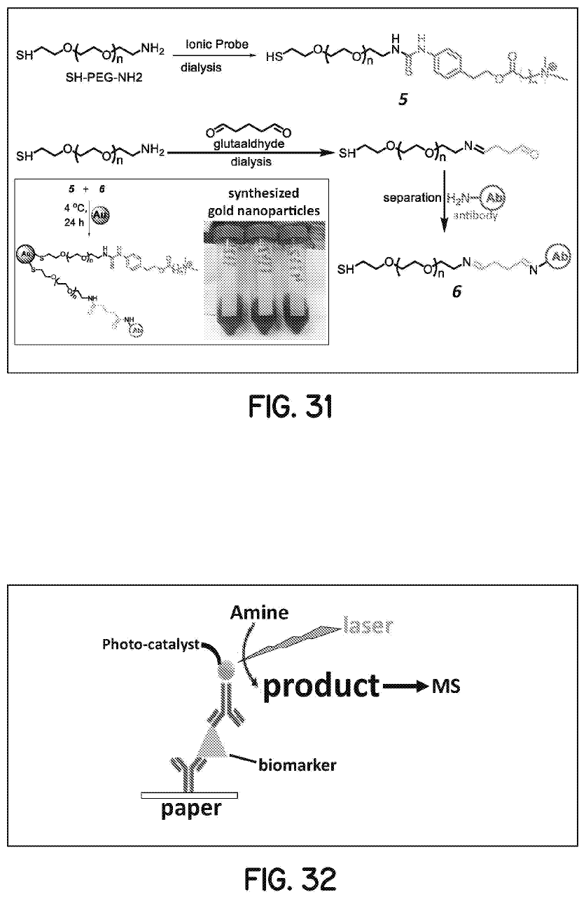

In other embodiments, gold nanoparticles that contain well-defined cleavable ligands at their surfaces can be deployed in the reporter antibody. The procedure for preparing the active ligand-bound gold nanoparticles is summarized in FIG. 31. The bi-functional PEG polymer (HS-PEG-NH₂) can be employed to anchor both the reporter compound and the antibody to the gold nanoparticles. A cross-linker, for instance, glutaraldehyde, will be used to couple the antibody to HS-PEG-NH₂ yielding product 6. It is preferred to have an excess of reporters on the gold nanoparticle compared with antibody, for instance by utilizing an excess amount of product 5 over 6. All cleavable modes (i.e., pH change, UV illumination, and redox chemistry) can be employed for this signal amplification approach. Gold nanoparticles in three different sizes (15 nm, 25 nm, and 40 nm) have been prepared by controlling the ratio of HAuCl₄ and sodium citrate (insert, FIG. 31).

In other embodiments, the reporter antibody includes a photoredox catalyst component. The presence of the sandwich complex in the system can be determined by introducing a compound known to react when irradiated in the presence of the photocatalyst. In some cases the irradiated can be exposure to visible light, while in other cases a dedicated light source, e.g., a laser or flashlight can be employed. Exemplary photoredox catalysts include Rose Bengal, Eosin Y, TPP⁺, Mes-Acr⁺, and riboflavin type systems. A suitably functionalized photoredox catalyst may be conjugated to an antibody using conventional chemistries. In one embodiment, after formation of the sandwich complex and removal of the unreacted reported antibody, triethanolamine is introduced to the substrate, which is converted to diethanolamine by the photoredox catalyst. Subsequent MS analysis can be used to detect the presence of diethanolamine, thus indicating the presence of the sandwich complex. In some analytical settings, the presence of esterases in certain blood sample can cause cleavage of the ester bond during assay. In such cases, the photoredox process or other pH-active functional groups (e.g., hydrazones, oximes, etc.) can be used as part of the structure of the probe to reduce esterase and other biological effects.

The following four steps can be used to prepare devices for the disclosed assay: (1) Paper Oxidation—oxidization of hydroxyl groups in cellulose to aldehyde groups—suitable methods include soaking the paper in 0.031 M KIO4 solution and heating to 65° C. for 2 hours; (2) Wax-Printing—either before or after aldehyde functionalization, the paper can be dried, and the working/sensing test zones are created by solid wax printing, for instance to form circular hydrophobic barriers on the paper substrate. The wax printing process produces hydrophobic barriers that extend through the thickness of the paper and effectively confines aqueous test reagents; (3) Covalent Antibody Binding on Paper; and (4) Blocking—empty sites in the paper test zones are blocked with Tris to prevent analyte non-specific binding.

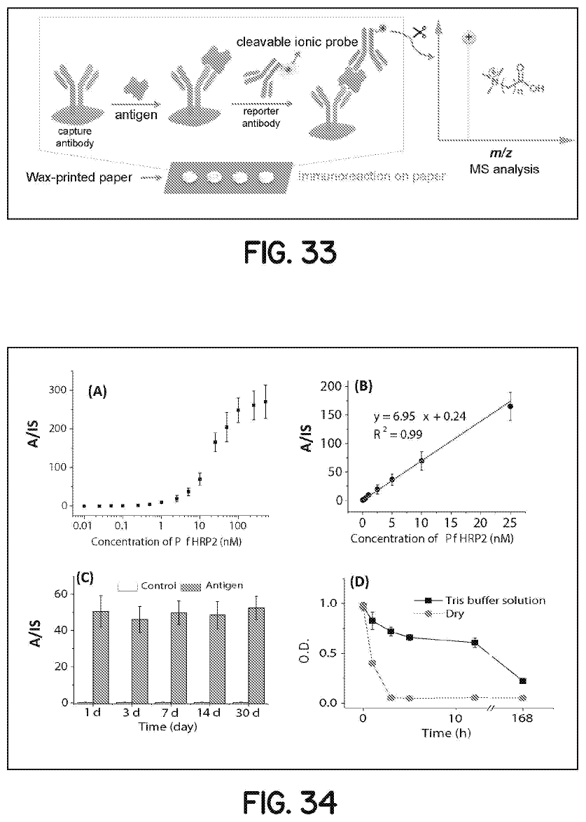

By immobilizing a specific antibody that recognizes a particular disease biomarker, the resultant paper surface becomes a bioactive sensing device that can be used for the immunoassay (see FIG. 33).

Antigen capture: For the immunoassay step, a solution (e.g., blood, saliva) containing a target antigen (for instance PfHRP-2 and/or P. aldolase as malaria biomarkers) are added to the bioactive paper surface containing the immobilized antibody that recognizes a specific epitope on the biomarker. After incubation, the test zones are washed, for instance one or more times with PBS buffer.

The reporter antibody is then added to the paper. The binding of the reporter antibody to the antigen immobilizes the reporter antibody to the paper. A buffer wash step will remove unbound antibody.

Following the capture of analyte and reporter antibody, the sandwich complex can be treated to release the reporter compound. For hydrolytically labile linkers, a drop (5 μL) of an aqueous NH₄OH basic solution will be applied to the paper test zones to release the reporter compound, which will be detected using wax-printed on-chip paper spray MS. Apart from the washing step, no purifications or amplifications are needed prior to analysis.

Another aspect of the present invention provides a simple test apparatus and method that allows an individual to perform the sandwich immunoassay in an in-home setting, permitting early disease detection in a manner that will be faster, simpler, and cheaper than those currently available and will be more sensitive for early stage detection, less susceptible to false positive/negative outcomes, and technologically flexible allowing the process to be readily refined as new biomarkers become viable. In some embodiments, the apparatus includes a paper substrate functionalized with a capture antibody, and a reporter antibody suitable formulated to be combined with the capture antibody following treatment with blood. In certain embodiments, the paper substrate is in direct contact with a finger stick, enabling direct transfer of blood to the paper substrate. After the patient or caregiver adds the reporter antibody formulation to the paper substrate, the apparatus can be sent to a lab for processing.

This platform may use antigen/antibody interactions for biomarker capture from biofluids, followed by on-chip MS detection. As a result, this aspect of the present invention may provide three unique levels of testing: (1) point-of-care (POC) application, (2) community-based surveillance detection—useful in a contagious disease setting (e.g., malaria) to identify people with latent infection that serve as reservoirs for continuous transmission of the disease, and (3) field analysis in the case of an outbreak (which typically occurs every rainy season in endemic regions).

Also disclosed is a 3D analysis platform including multiple layers. The platform includes at least a reagent layer containing the reporter antibody, and a capture layer containing the capture antibody. The biological sample is deposited above the reagent layer, through which it diffuses, binding the antigen to the reporter antibody. The complex then passes to a capture layer, where it is immobilized by the capture antibody conjugated to the substrate. After removal of remaining biological sample and unreacted reporter antibody (for instance by washing/immersing in an aqueous solution), the reporter compound can be obtained from the sandwich complex as described above. The layers can be bound together by double sided adhesive tape to enable easy separation of individual layers for subsequent on-chip MS detection by paper electrospray MS. In some embodiments, there can be a dwell layer disposed between the reagent layer and the capture layer, providing additional time for the formation of the antigen-reporter antibody complex as the biological sample passes through the platform. In certain embodiments, the platform can include a plasma separation layer disposed upstream of the reagent layer to filter cellular components of the biological sample prior to contacting the reagent layer. The platform can include a paper detection layer disposed downstream of the capture layer. The detection layer can include a modified cellulose, a tip and directing channel as described above. In some embodiments, the reagent layer can include a plurality of different reporter antibodies, each specific for a different antigen and releasing a different reporter compound. In some instances, the platform can include channels defined by wax or other impermeable material to guide the biological sample through the platform. The platform can include a splitter layer disposed upstream of the reagent layer, and generally after the plasma separation layer, which divides the biological sample and directs each portion to a different segment of the reagent/capture layers, each segment containing a different reporter/capture antibody pair. Platforms including a splitter may also include a collimating layer upstream of the detection layer, which rejoins the divided portions of the sample prior to analysis. Although the detection layer should be cellulose-based in order to facilitate paper-spray mass spectrometry, the remaining layers of the system can be materials other than cellulose. Exemplary materials are disclosed by D. Kim et al., in Protein immobilization techniques for microfluidic analysis, Biomicrofluidics (2013) 7, 041501, the contents of which are hereby incorporated by reference.

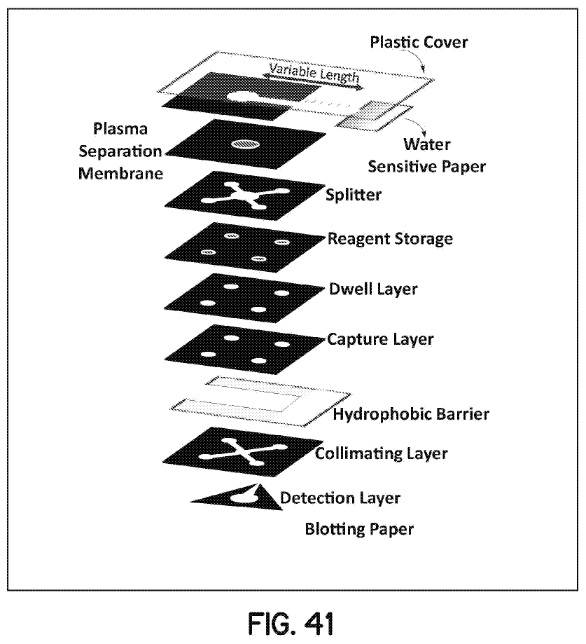

An exemplary platform is depicted in FIG. 41; black regions: hydrophobic wax barrier; white regions: hydrophilic test paper zones). The volume of the sample (e.g., finger prick blood) will be determined by the topmost layer and quartered upon reaching the splitter. In an exemplary embodiment, A33 and CEA reporter antibodies for CRC will be in the reagent layer and corresponding capture antibodies conjugated to the capture layer. Two test zones in the capture layer permit simultaneous A33 and CEA detection, and the remaining zones act as positive and negative controls.

### EXAMPLES

The following examples are for the purpose of illustration of the invention only and are not intended to limit the scope of the present invention in any manner whatsoever.

### Example 1: Paper-Based Dried Blood Spheroid Collection for Stabilization of Labile Substances

Hydrophilic filter paper was converted into a hydrophobic paper substrate (thus having a lowered surface energy) by exposing the filter paper to headspace vapor of trichloro(3,3,3-trifluoropropyl)silane under vacuum in a desiccator. Because this approach utilizes a gas-phase preparation procedure, many of the physical/chemical characteristics (e.g., color, weight, porosity, tensile strength, malleability, flammability) of the filter paper remain unchanged. However, wettability of the paper is altered controllably by varying silane vapor exposure time.

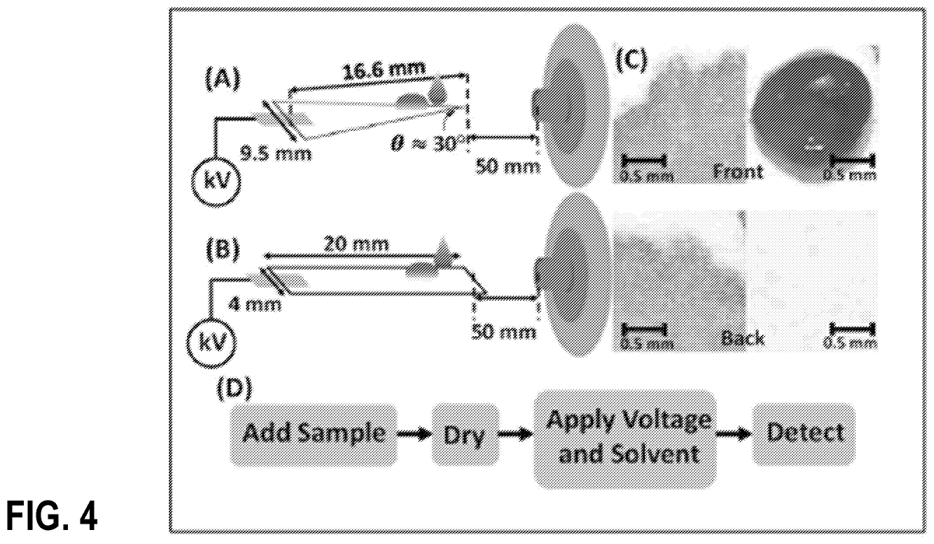

As a result of the lowered surface energy, aqueous-based samples such as blood, serum, and urine bead when applied onto the hydrophobic paper, and as a consequence, form 3D spheroids (molds) when allowed to dry (FIG. 4, panel c). As a result, only the outermost layer of the dried spheroid is exposed to air during storage, thereby preserving the integrity of majority of analytes inside the dried blood.

As observed from FIG. 4, panel c, the fresh blood penetrated to the back of the untreated paper forming a blood spot of area 0.13±0.05 cm², with >2× relative standard deviations between samples. In contrast, no traces of blood were observed on the back of the hydrophobic paper; instead the entire 4 μL blood volume rested on top of the hydrophobic paper strip, confined to a reproducible area of 0.017±0.003 cm′. Additionally, the whole dried blood spheroid could be punched for subsequent extraction without regard to volcanic, chromatographic hematocrit effects.

Two shapes of hydrophobic paper strips were used and tested [triangular and rectangular (FIG. 4, panels A and B); pre-cut before silanization] to investigate the effect of dedicated tips during direct, in-situ MS analysis of the dried blood spheroids. The overall workflow for blood collection and analysis from hydrophobic paper is as illustrated in FIG. 4, panel D, where 4 μL of blood was deposited onto the hydrophobic paper strip, dried for a specified time, and analytes were detected using hydrophobic PS MS.

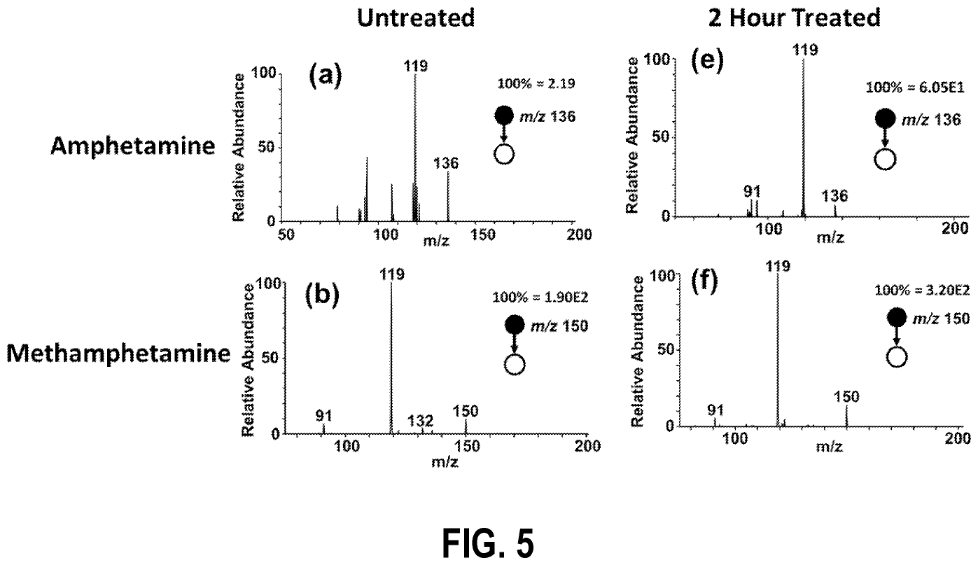

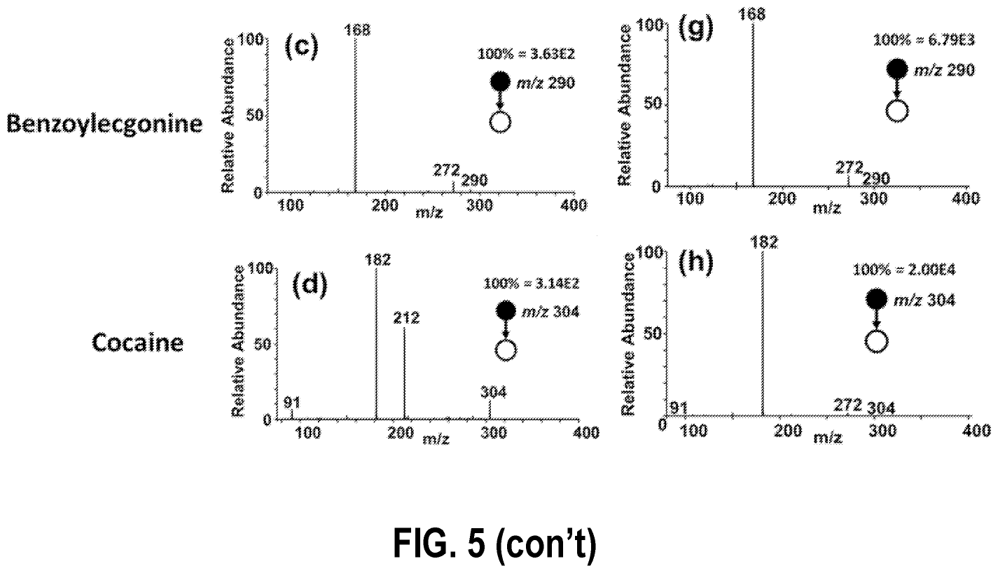

Using ethyl acetate as spray solvent, small organic compounds (e.g., amphetamine and methamphetamine) were selectively extracted and detected from blood and neat water-based samples dried on hydrophobic paper rectangles (FIGS. 5-7), albeit lower ion intensity compared with hydrophobic paper tringles due to the absence of a dedicated macroscopic tip. In comparison to untreated paper rectangles, however, an enhancement (>10×) in ion yield was observed when using the hydrophobic paper, with signal increasing with paper hydrophobicity (FIG. 5). In the absence of a dedicated sharp tip, electrospray occurs from randomly oriented fibers protruding from the edges of the paper. For untreated paper strips, these fibers easily bundle up, reducing the electric field needed to support electrospray-like ionization. The reduced wetting on hydrophobic papering turn decreases the probability of fiber collapse, providing individual fibers that support ionization with 3 kV of direct current (DC) voltage (FIG. 8).

Mass spectra were recorded using Thermo Scientific Velos Pro LTQ linear ion trap mass spectrometer. Dry hydrophobic PS spray plumes and vibrations were observed using a Watec camera (WAT-704R). Contact angles were observed using a Rame-Hart goniometer. Standard solutions (1.0 mg/mL) of benzoylecgonine, cocaine, amphetamine, and (±)-methamphetamine were obtained from Cerilliant (Round Rock, Tex.). All solvents were purchased from Sigma-Aldrich (St. Louis, Mo.). Human blood was purchased from Innovative Research (Novi, Mich.). Whatman filter paper (24 cm, grade 1), polycarbonate, and ethyl acetate membrane filters were purchased from Whatman (Little Chalfont, England). Using a digital template, paper triangles were cut from filter paper with an Epilog Legend36EXT laser.

Illicit Drug Analysis in Dried Water Samples on Paper Rectangles. 500 ng/mL of cocaine, benzoylecgonine, amphetamine, and methamphetamine were spiked into water and then dried onto the paper strip surface that was untreated or previously treated for 2 hours with silane. 3 kV and 20 μL of ethyl acetate were applied, and collision-induced dissociation (CID) of the target drugs was performed. Resulting spectra from untreated paper strips were typically 10× lower intensity than 2 hour treated paper. Results are shown in FIG. 5.

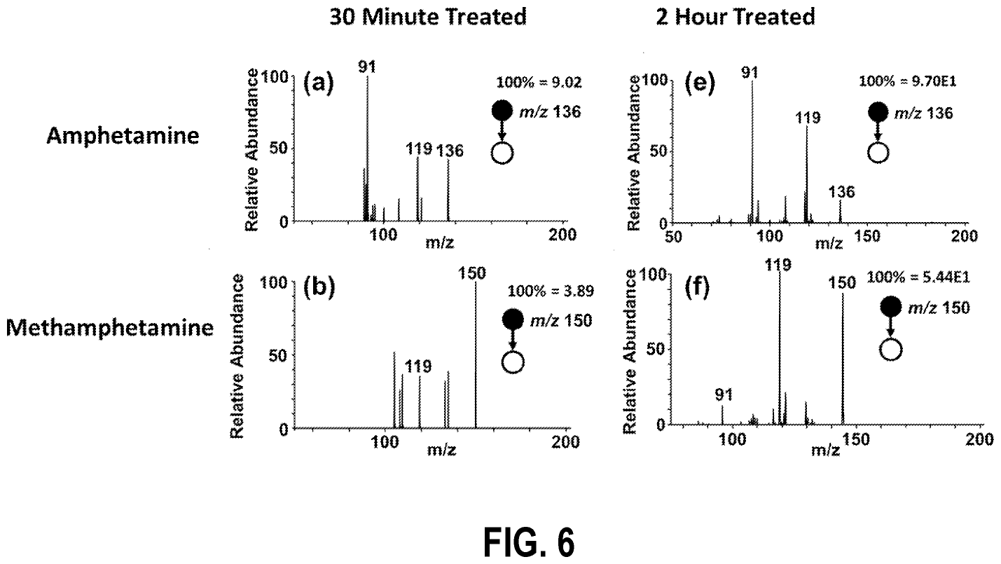

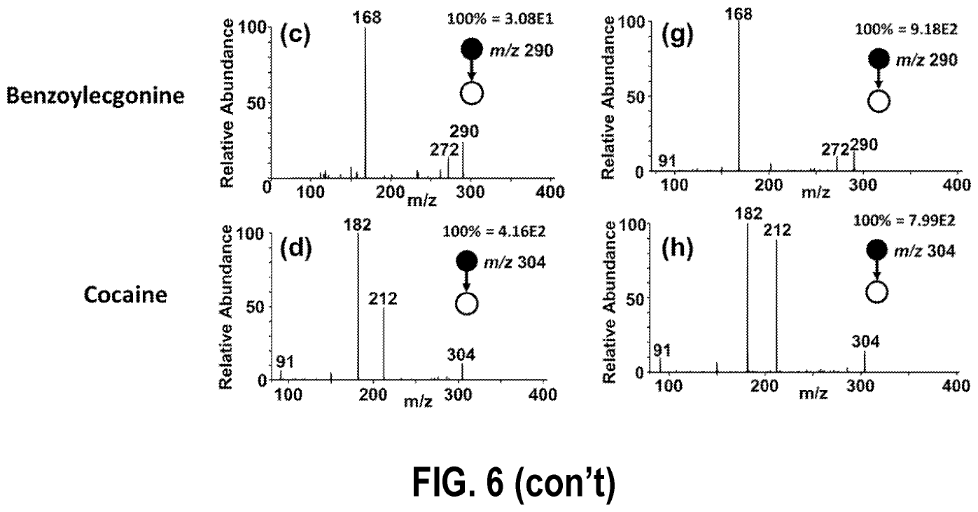

500 ng/mL of cocaine, benzoylecgonine, amphetamine, and methamphetamine were spiked into whole human blood and then dried onto the paper strip surface that was untreated or previously treated for 30 minutes or 2 hours with silane. 3 kV and 20 μL of ethyl acetate were applied, and CID of the target drugs was performed. Untreated paper strips showed no characteristic fragments of the target drugs, and therefore were excluded. This is due to increased matrix effects present from the blood in addition to the increased drug binding to the paper surface when compared to treated paper. Untreated paper is not able to overcome the matrix effects during extraction and also ionizes the analytes to a lesser extent. Treated paper performs a more efficient extraction and ionization step, so the target analyte is observed. Results are shown in FIG. 6.

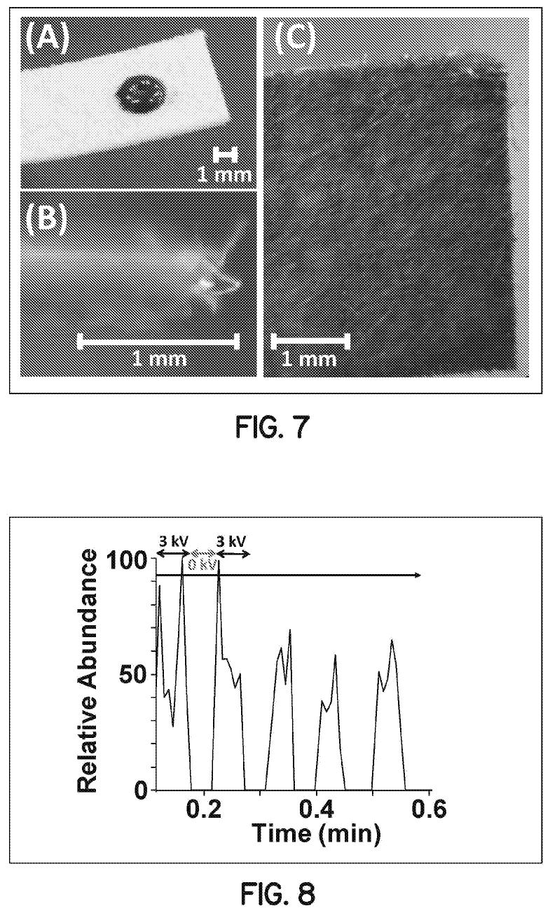

Referring to FIG. 7, pictures of the blunt edge of the paper strips were taken with a Watec camera for close-up images. Although a blunt edge is expected to not produce ionization, loose paper fibers protrude from the end of the strip. These fibers are expected to allow Taylor cones to form when sufficient solvent and high voltage is applied. This spray process is facilitated from treated paper where wetting is reduced, freeing individual fibers for electrospray.

In order to determine the basis of ionization, whether it be from pressure difference at the mass spectrometer (MS) inlet or from applied voltage, 3 kV (against the grounded MS inlet) was applied to the paper strip for a short time. The voltage was then changed to 0 kV. Referring now to FIG. 8, the total ion chromatogram shows the signal is only present at times when voltage is applied to the paper strip. This process shows that ionization is dependent on the applied voltage, and therefore the most likely method of ionization through electrospray-like mechanism from the paper strip. Because no tip is present on the strip (such as one present on paper triangles), ionization most likely occurs when a Taylor cone is formed on individual paper fibers that protrude from the blunt end of the paper strip.

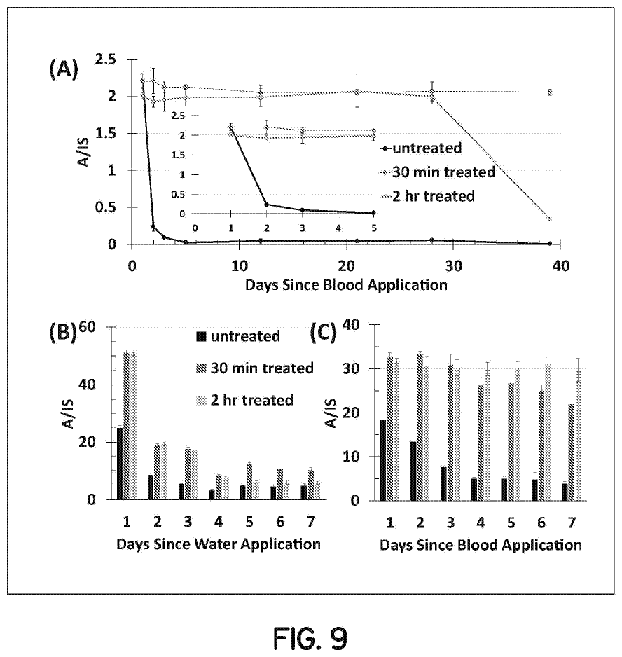

Cocaine and diazepam (2 μg/mL) were spiked separately into whole human blood, and 4 μL aliquots were spotted onto the as-prepared hydrophobic paper and stored in ambient air for a maximum of 40 days. Similar blood samples were stored using the conventional DBS method on untreated, hydrophilic paper strips. Results from these analyses are summarized in FIG. 9, which indicate that both cocaine (FIG. 9, panel A) and diazepam (FIG. 9, panel C) trapped inside the 3D dried blood spheroid are stabilized compared with storage done under the porous DBS conditions. About 90% of cocaine is hydrolyzed in just the next day of blood storage on untreated hydrophilic paper (insert, FIG. 9, panel A). Significant cocaine oxidation was observed after 40 days of storage under the dried blood storage conditions.

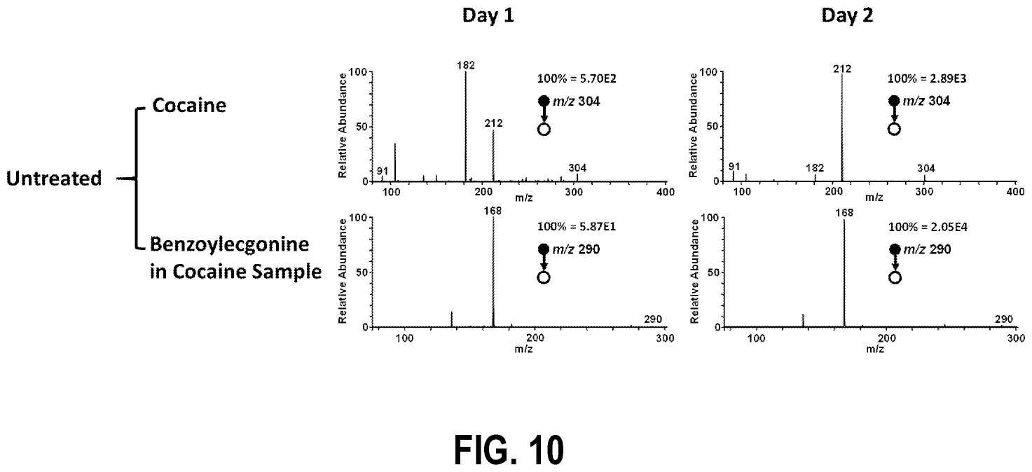

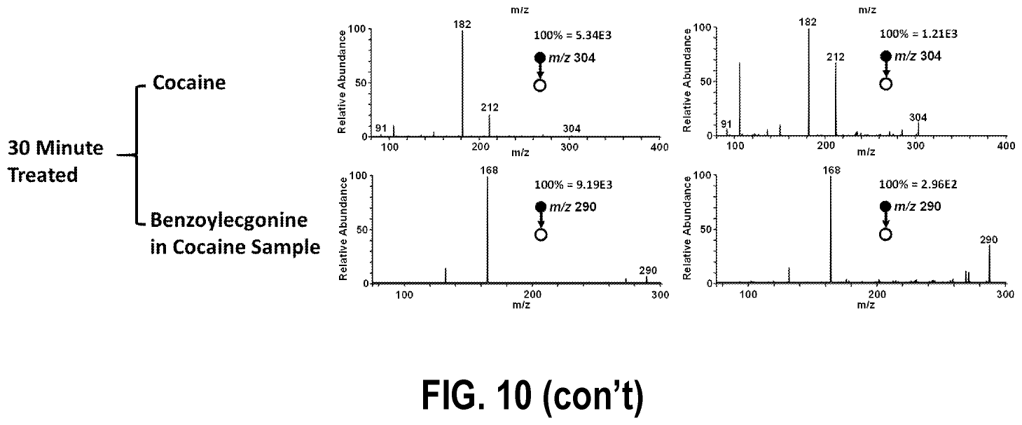

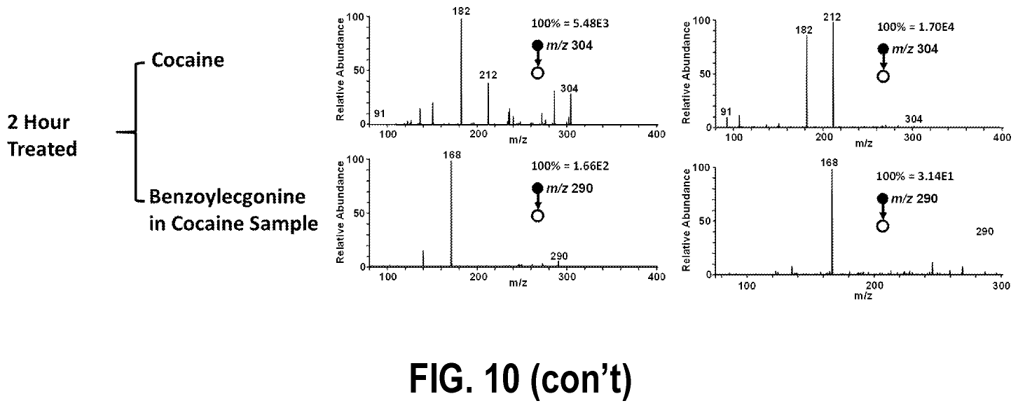

Benzoylecgonine is a metabolite of cocaine, but benzoylecgonine can also be a degradation product of cocaine. This degradation could be the cause of decrease of cocaine intensity found in FIG. 9. To monitor this, an offline extraction of the dried blood spots/spheroids in ethyl acetate was performed and analyzed via nanospray. Between days 1 and 2, benzoylecgonine intensity found in the cocaine-spiked blood sample increased relative to the cocaine intensity, indicating cocaine likely degraded to become benzoylecgonine while the sample was stored. Paper treated for 30 minutes and 2 hours did not experience this sharp increase in benzoylecgonine. These results are shown in FIG. 10.

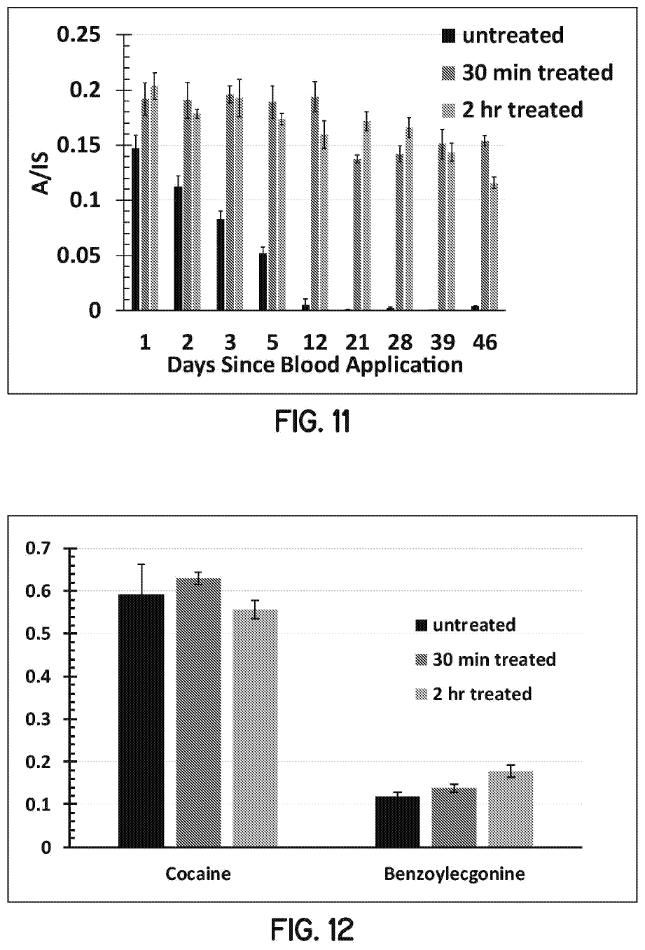

The stability of benzoylecgonine stored on treated versus untreated was compared. Benzoylecgonine was found to degrade faster on untreated paper compared with treated paper substrates. Compared with cocaine degradation (FIG. 9, panel A), however, benzoylecgonine was relatively more stable on untreated paper lasting for at least 12 days before complete decomposition, compared with the 2 days for cocaine (these results are shown in FIG. 11). Here, greater than 95% of benzoylecgonine was oxidized (via the direct addition of oxygen) after the 12th day of storage under the typical DBS condition. In contrast, a stable ion signal was detected for benzoylecgonine stored in the spheroid even after the 46th day. Unlike cocaine, the signal loss for diazepam on DBS was gradual, and 21% of the analyte remained in DBS after one week (untreated, FIG. 9, panel C). As expected, signal was relatively stable when stored using the dried blood spheroid methodology.

In order to confirm oxidative degradation as the cause for instability of cocaine and benzoylecgonine in blood spots/spheroids, samples identical to those featured in FIG. 9, panel A, and FIG. 11 were stored in a vacuum desiccator for 15 days and then analyzed with 10 μL of 500 ng/mL deuterated internal standard in ethyl acetate. Results show that cocaine and benzoylecgonine are stable under this airtight storage condition, irrespective of the type of paper (see FIG. 12). This confirms analyte degradation observed in FIGS. 9 and 11 are due to oxidative stress from air.

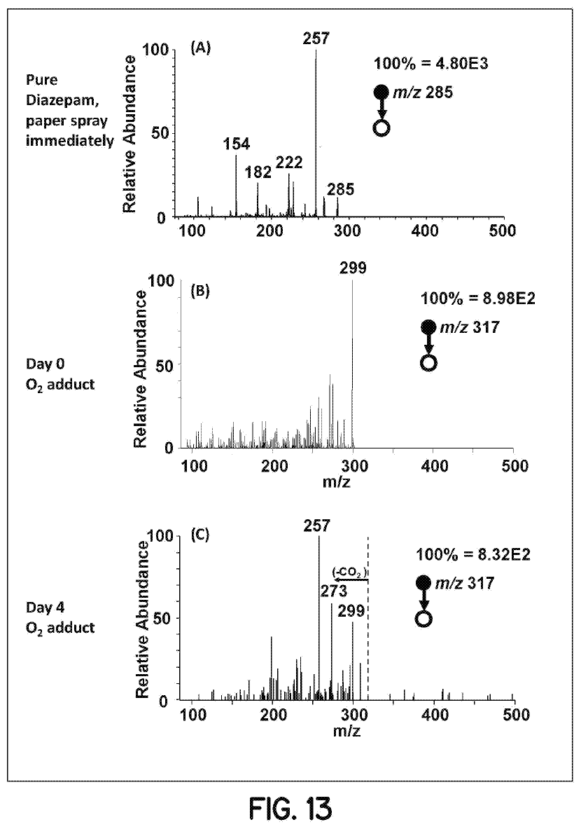

The involvement of oxygen is also confirmed by the direct detection of O₂ adducts (+32 Da increase) (FIG. 13). Neat diazepam was deposited onto paper triangles and analyzed immediately (day 0) or after 4 days of ambient storage. Lack of signal from the +32 peak (FIG. 13, panel B) on day 0 compared with signal on day 4 (FIG. 13, panel C), which yields water loss (m/z 299), CO₂ loss (m/z 273), and a common ion with pure diazepam (m/z 257). (FIG. 13, panel A). This indicates an increase in O₂ addition to diazepam as it rests in ambient conditions for several days, contributing to the decreasing intensity of diazepam noted in FIG. 9, panel B and panel C. To investigate a possible “wall effect” in the stabilization of analytes in dried blood spheroids, the stability of neat, dry diazepam (prepared in water, as opposed to blood) on both treated (no spheroid was formed) and untreated paper substrates were compared, and found to be similar (FIG. 9, panel B). That is, neat diazepam analytes gradually degraded at comparable rates on both hydrophobic and hydrophilic paper strips. Ion intensities recorded from treated paper strips were relatively higher than those from untreated paper because of higher ionization efficiency of hydrophobic paper substrates. Collectively, these results suggest that the creation of 3D spheroid from a viscous sample like blood is essential in preventing oxidation in air, and that the interior of the spheroid was protected by providing a possible critical radius of insulation that increases the spheroid's resistance to thermal conduction and oxidative degradation. Finite element analysis of thermal energy flux from surrounding ambient air for spheroid and DBS approximated geometry confirms the spheroid's enhanced thermal protection over a given time period. The reduced surface area-to-volume ratio of the spheroid limits bulk exposure to the ambient environment (FIG. 9), which also improves resistance to oxidative degradation over time.

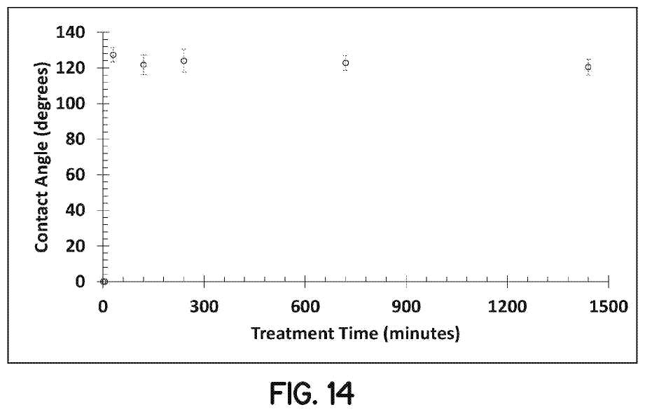

Contact angle measurements with water is the most prominent method used in estimating surface energies of planar substrates. However, for porous and rough surfaces such as paper, contact angle measurements yielded inconsistent results (e.g., FIG. 14 shows contact angles of ˜125° for any treatment longer than 5 minutes). In this part of the investigation, DI water was deposited on the surface of filter paper treated for 0, 5, 30, 120, 240, 720, and 1440 minutes. The contact angle of this water drop was observed using a Rame-Hart goniometer. The contact angle corresponds to the relative surface energy per area of the paper when compared to the surface tension of the water. If the surface energy of the paper exceeds the surface tension of the water (72 mN/m), the water will completely wet the paper and the contact angle will be 0°. If the surface energy does not exceed the surface tension of the water, the water drop will bead up, and the contact angle between the water drop and the paper will be some angle θ.

As seen in FIG. 14, the contact angle θ was 0° for untreated paper and paper treated for 5 minutes. For paper treated for 30 minutes or greater, the contact angle θ was approximately 125°. These results indicate that paper treated for 5 minutes or less have a surface energy per area greater than 72 mN/m.

Because contact angle measurements yielded inconsistent results, the present inventors developed a novel electrostatic spray-based method in which a simple multimeter is used to measure total ion current or via selected ion monitoring by MS. Here, solutions consisting of water/acetonitrile mixtures were prepared. The specific proportion of water/acetonitrile used was varied, which yields known surface tensions for each solution prepared (as shown in Table 2, below). Acetonitrile/water mixture surface tensions were originally determined by Rafati et. al. (J. Chem. Eng. Data, 2010, 55, pp. 4039-4043). Table 2. Reported surface tensions of acetonitrile/water mixtures.

| | Mole | Mole | Does the | Literature |
|---|---|---|---|---|
| | Fraction | Fraction | solvent wet | Surface |
| Solvent | of | of | 2-hour treated | Tension |
| number | Acetonitrile | Water | paper? | (mN/m)[2] |
| 1 | 0.0149 | 0.9851 | No | 62.36 |
| 2 | 0.0298 | 0.9702 | No | 55.92 |
| 3 | 0.0516 | 0.9454 | No | 49.39 |
| 4 | 0.0950 | 0.9050 | No | 40.54 |
| 5 | 0.1227 | 0.8773 | Partially | 37.97 |
| 6 | 0.2541 | 0.7459 | Yes | 32.92 |
| 7 | 1 | 0 | Yes | 29.3 |

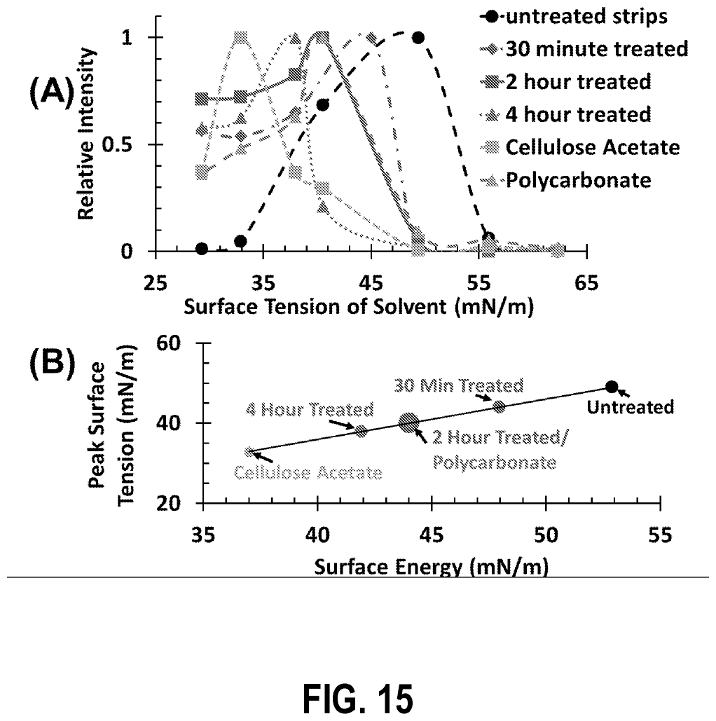

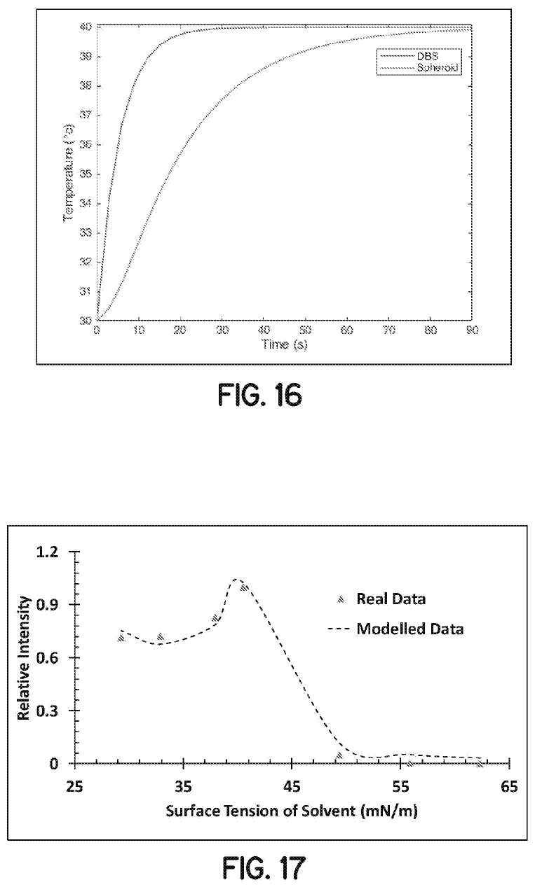

These solution mixtures were used as spray solvents in electrostatic spay where neat benzoylecgonine analyte dried on the hydrophobic paper was ionized in the process. Because the electrostatic spay ionization of dry samples is a function of solubility and wettability, the present inventors anticipated a maximum ion signal to be recorded when the surface tension of the spray solvent approximately equals the surface energy of the hydrophobic paper. This expectation has been met (FIG. 15, panel A). Maximum/peak ion currents were observed at solvent surface tensions of 38, 40, and 44 mN/m for hydrophobic paper substrates prepared by 4 h, 2 h, and 30 min silane exposure times, respectively. Polymeric membranes of known surface energies were also employed: cellulose acetate (37 mN/m) and polycarbonate (44 mN/m). The corresponding peak currents were observed at 33 and 40 mN/m (FIG. 15, panel A), respectively, which correlated well with the known surface energies of the membranes. The position of the peak current can be used to determine the surface energy of the paper/membrane from which the electrostatic spray is derived. Therefore, a calibration curve was subsequently constructed using the two membranes as standards and plotting the known and the experimentally determined surface energies (FIG. 15, panel B). Through this calibration, the surface energies of the as-prepared hydrophobic paper substrates were estimated to be 42, 44, and 48 mN/m for 4 h, 2 h, and 30 min treatment times, respectively (see Table 3, below). Samples of treated paper were used to ionize dried 2 μg/mL benzoylecgonine using solvents found in Table 2. Measured relative ion intensities are shown in FIG. 15; extracted surface energies are summarized in Table 3. These results agree with the surface energy reported for cellulose acetate and polycarbonate standards [see Damon, D. E., et al., Anal. Chem., 2016, 88, pp. 1878-1884]. (Further, heat transient simulation analysis—as shown in FIG. 16—showed that both blood storage geometries had an initial temperature of 30° C., and were subject to a constant ambient air temperature of 40° C.)

TABLE 3
Surface energy determination using peak surface tension solvent.

| | Reported | Peak Surface | Calculated |
|---|---|---|---|
| | Surface | Tension | Surface |
| Surface name | Energy (mN/m) | Solvent (mN/m) | Energy (mN/m) |
| Cellulose Acetate | 37 | 33 | — |
| Polycarbonate | 44 | 40 | — |
| 4 Hour Treated | — | 38 | 42 |
| paper | | | |
| 2 Hour Treated | — | 40 | 44 |
| paper | | | |
| 30 Minute Treated | — | 44 | 48 |
| Paper | | | |
| Untreated Paper | — | 49 | 53 |

These experimentally determined surface energies were validated through a theoretical modeling (Equation S2—below) based on analyte partitioning and a residual difference between solvent surface tension and paper surface energy. The theoretical Basis for Surface Energy Estimation via Electrostatic spray using Solvents with Different Surface Tensions can be explained as follows: Optimum ion current is expected when solvent surface tension is approximately equal to the surface energy of the paper/polymer surface. Therefore, evaporation rate post-Taylor cone is negligible in determining the solvent surface tension that yield highest ion signal, when compared to the effect of wetting and slight partitioning effects. Three regions in FIG. 15, panel A, can be distinguished: (1) region before the maximum current, involving solvents with lower surface tension than surface energy of the surface, (2) the point at which the ion current is maximum or peaks; the corresponding solvent surface tension is expected to equal the surface energy of the paper substrate, and (3) region after the peak current where solvent surface tension is great that surface energy of paper.

If the partition coefficient is defined as:

K=Cₛₒₗᵥₑₙₜ/Cₚₐₚₑᵣ (Equation 1)

where Cₛₒₗᵥₑₙₜ is the concentration of the target analyte in the spray/extraction solvent, and Cₚₐₚₑᵣ is the concentration of the target analyte on the paper surface, then, each of the regions will have the following properties: (1) Low surface tension solvent, high degree of wetting, high degree of paper-solvent analyte interaction resulting in possible redistribution of analyte back into the paper substrate post-extraction, high evaporation rate (because of spreading), very small K value (2); Solvent for the peak ion current must have surface tension that allows intermediate wetting, less paper-solvent-analyte interaction and less analyte re-deposition post-extraction. It will also have moderate evaporation rate and large K value; (3) High surface tension solvent, low degree of wetting, low degree of paper-solvent analyte interaction resulting in low amount of extraction and low amount of re-deposition, low evaporation rate, and small K value.

Using this logic, and using fitting parameters to correct for a changing K value, the inventors have determined the following equation to account to the shape of the ion current observed in FIG. 15, panel A:

$$I \approx \frac{aK}{\vert {SE}^{2} - {γ}^{2} \vert + b γ - c} \qquad ( Equation 2 )$$

where I is ion intensity; a, b, c are fitting parameters; K is the partition coefficient; SE is the surface energy of paper; and γ is the surface tension of solvent.

By setting K to 1 and SE as 44 mN/m, the present inventors fitted this equation to the data collected for 2 hour treated paper and the result is shown in FIG. 17, showing a good fit between theoretical and experimental data.

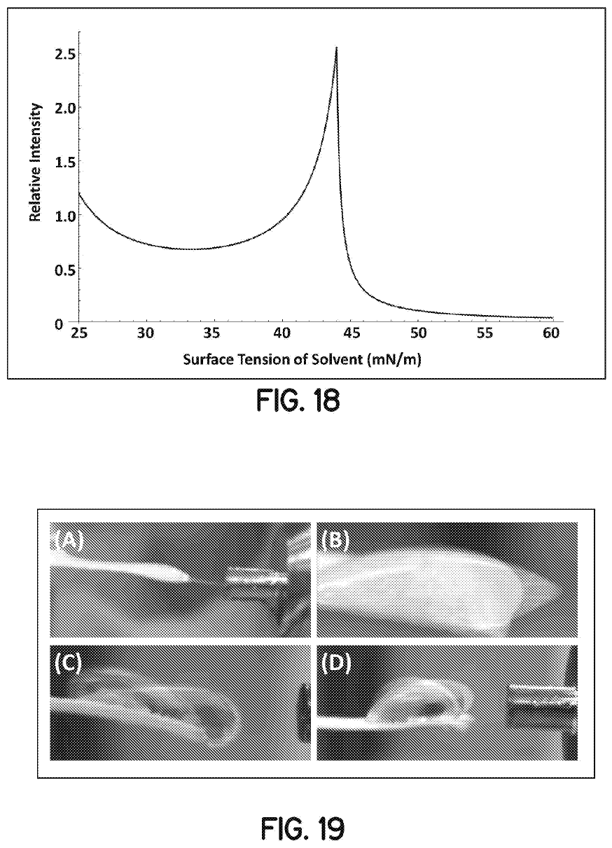

Excellent fitting was obtained only when using the calibrated surface energies and not the position of the peak current (FIGS. 17 and 18). The shape of this function, when plotted with these same fitting parameter values, is shown in FIG. 18. This shape/function implies there is an actual peak surface tension that is approximately equal to surface energy of the paper and is possible to be found experimentally. 20 μL of acetonitrile/water solutions of varying ratios were deposited onto the front of paper strips that had been previously treated for 2 hours each with trichloro(3,3,3-trifluoropropyl) silane. 5 kV DC voltage was applied to the back of the paper strip (away from the solvent), and the strip was pointed toward a mass spectrometer inlet. (FIG. 19 includes still shots from videos of this process).

The present inventors note that some of the acetonitrile/water solvent mixtures used for surface energy estimations did not wet the hydrophobic paper, and yet ions were detected. Under this non-wetting condition, an electrostatic spray mode has been proposed [by Damon, D. E., et al., Anal. Chem., 2016, 88, pp. 1878-1884] where capacitive charging at droplet surface causes analyte ions to oscillate. Ions break free from the liquid droplet surface at a sufficiently high kinetic energy, determined by the applied DC voltage (onset voltage determined to be 3 kV). To evaluate the effect of surface tension of the solvent on this electrostatic spray mode, a camera was used to image the spray dynamics of solutions comprising of varying mole ratios of acetonitrile in water. FIG. 19 reveals droplet oscillation was reduced with decreasing solvent surface tension, from FIG. 19, panel C to FIG. 19, panel A, at which point spray mode becomes electrospray. The corresponding ion current data (FIG. 15, panel A) indicate low abundance of ion yield was recorded for both very high and low surface tension solvents. This is because solvent with high surface tension is less likely to form a stable Taylor cone while low surface tension solvent suffers from unfavorable partitioning.

The observations of the present inventors show that as surface tension increases (with water being a larger constituent), the solvent beads onto the surface of the paper and vibrates when voltage is applied (Surface tension 62 mN/m); As surface tension of the solvent decreases (when the acetonitrile proportion grows), the solvent bead vibrates more violently (Surface tension 41 mN/m. An even greater amount of acetonitrile in the solvent will cause the solvent drop to both vibrate and form momentary Taylor cones (Surface tension 38 mN/m); when voltage is applied, the solvent vibrates, momentary Taylor cones are observed. Finally, when acetonitrile is a large enough constituent of the solvent, the solvent is able to completely wet the paper, and droplet vibrating ceases. Instead, a stable Taylor cone is observed (Surface tension 29 mN/m; when voltage is applied, the solvent forms multiple Taylor cones from the paper fibers protruding from the flat edge of the paper strip.

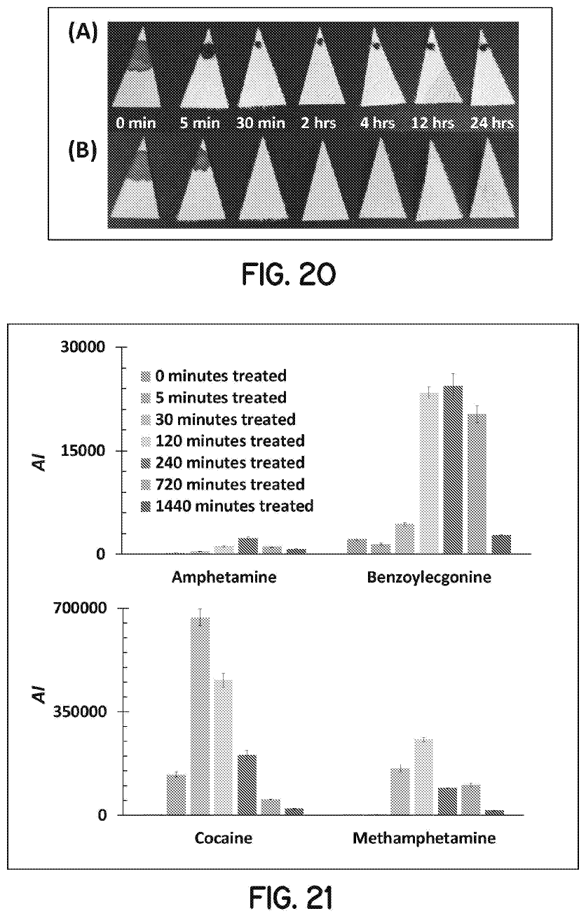

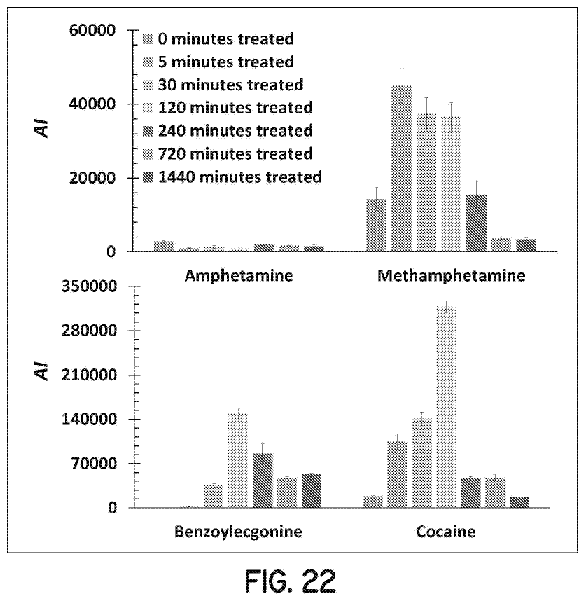

The quantitative abilities of the direct hydrophobic PS MS method was also assessed using dried blood spheroid samples containing amphetamine, methamphetamine, cocaine, or benzoylecgonine. The initial investigations involved the use untreated paper and hydrophobic paper triangles treated with silane vapor at 5, 30, 120, 240, 720, and 1440 min exposure times. These samples were analyzed with ethyl acetate as the spray solvent, and the absolute intensities of the fragment ions derived from collision-induced dissociation were quantified. Overall, the paper treated for 30 and 120 introduced the highest intensity responses and were selected for further testing (FIGS. 20-22).

More specifically, four illicit drugs: cocaine, benzoylecgonine, amphetamine, and methamphetamine in dried blood spots were extracted on paper triangles that were untreated and treated for 5, 30, 120, 240, 720, and 1440 minutes. Solvent used was 20 μl of ethyl acetate. Most abundant ions are present typically on 30-minute treated paper and 2 hour treated paper, so these treatments were used for analysis in the further studies.

Optimization of Treatment Time with Acetonitrile. Similar to the procedure used to obtain FIG. 21, the four illicit drugs cocaine, benzoylecgonine, amphetamine, and methamphetamine in dried blood spots were extracted on paper triangles that were untreated and treated for 5, 30, 120, 240, 720, and 1440 minutes. These responses were found to be influenced by i) drug affinity binding to the paper surface versus its solubility in the spray solvent (partitioning); ii) ionization efficiency—the impact of treatment time on PS performance; and iii) extraction efficiency of the analyte from the dried blood. Treatment time was not observed to affect analyte ionization due to similar wetting of ethyl acetate on all paper triangles, which produced protonated ions via electrospray-based mechanism (as opposed to electrostatic ionization)

Properties of the blood spheroids appeared identical (e.g. size and interaction with paper surface) on all paper treated for >30 min. Therefore, partitioning of the analyte between the paper and the solvent post-extraction is expected to be the major contributing factor affecting ion yields from treated paper substrates. This partitioning factor is in turn controlled by the log P of drug and paper treatment time (Table 4). For example, cocaine is the most hydrophobic drug tested (log P 2.28) and it was observed to have a higher ion intensity on paper with a shorter treatment time (i.e., less hydrophobic paper substrate: FIG. 21). Similarly, benzoylecgonine, the most hydrophilic drug tested (Log P−0.59), showed a higher ion signal on paper with a longer treatment time (i.e., more hydrophobic). These results may be explained by that fact that molecules with high log P values prefer hydrophobic medium (and vice versa), and thus binding capacity is small in paper substrates prepared by shorter treatment times, enabling enhanced ion yield from such surfaces.

TABLE 4
Limits of Detection (LOD) and Quantification
(LOQ) of drugs in dried blood on triangles

| | | LODs (LOQ)s in | | | | | |
|---|---|---|---|---|---|---|---|
| | | dried blood | | | | | |
| | | (ng/mL) | | | | | |
| | | Untreated | | 30 Minute | | 2 hour | |
| | | Paper | | Treated | | Treated | |
| Analyte | LogP | Triangle | | Triangle | | Triangle | |
| Amphetamine | 1.80 | 4.4 | (9.7) | 0.12 | (1.3) | 0.11 | (0.69) |
| Methamphetamine | 2.24 | 7.9 | (9.1) | 0.34 | (1.8) | 1.7 | (4.2) |
| Cocaine | 2.28 | 3.5 | (19) | 0.37 | (0.57) | 1.0 | (1.7) |
| Benzoylecgonine | −0.59 | 3.7 | (7.9) | 0.48 | (0.79) | 0.49 | (1.6) |

LODs were calculated from respective calibration curves using signal corresponding to (S_{blank}) + 3 × σ_{blank}; LOQs were calculated from respective calibration curves using signal corresponding to (S_{blank}) + 10 × σ_{blank}; where (S_{blank}) is the average blank signal and σ_{blank} is the standard deviation of the signal from 3 replicates.

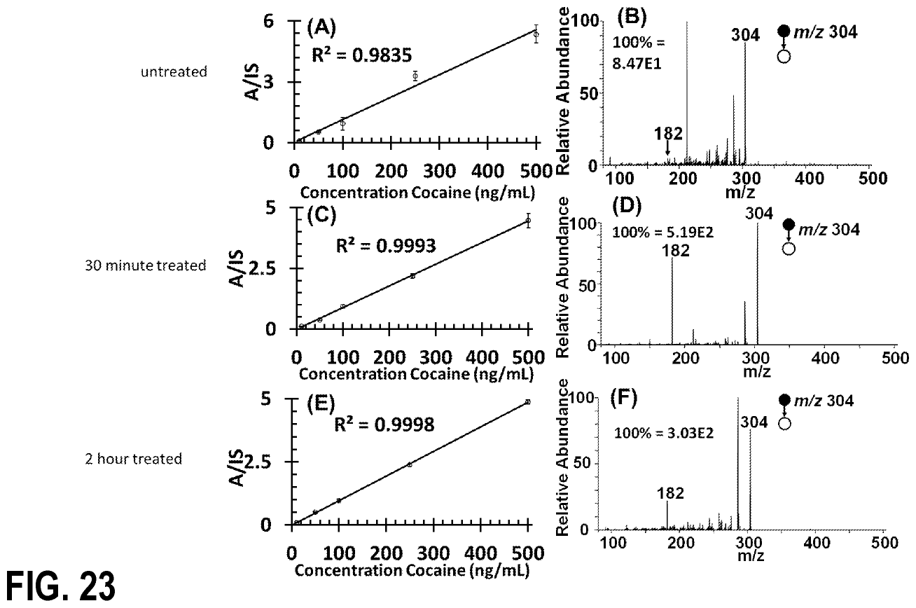

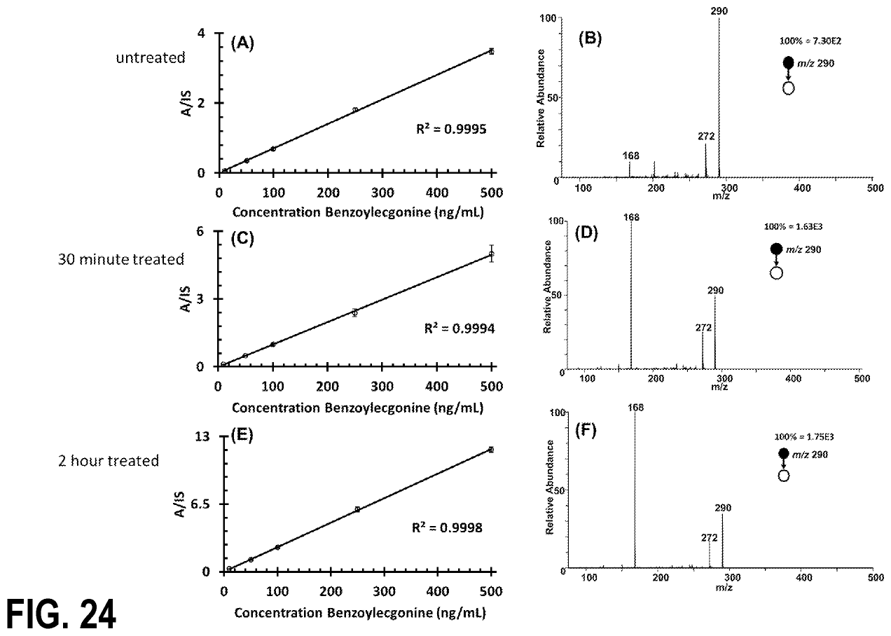

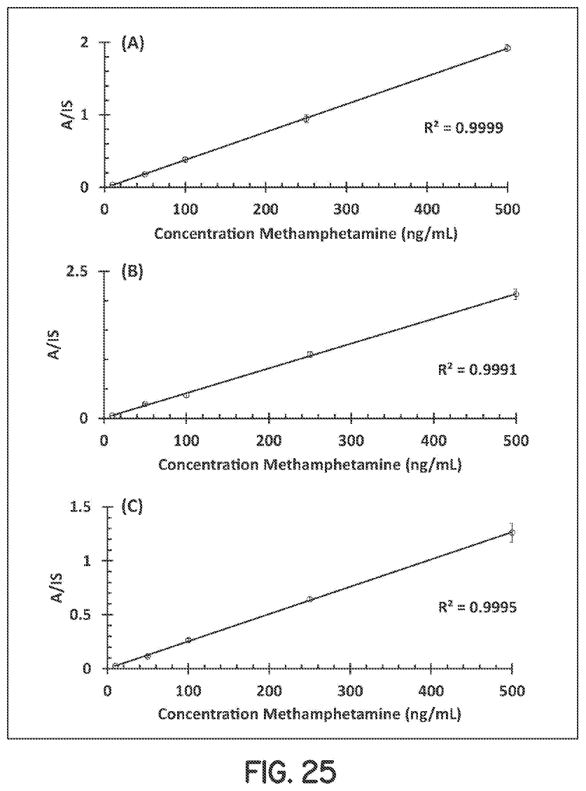

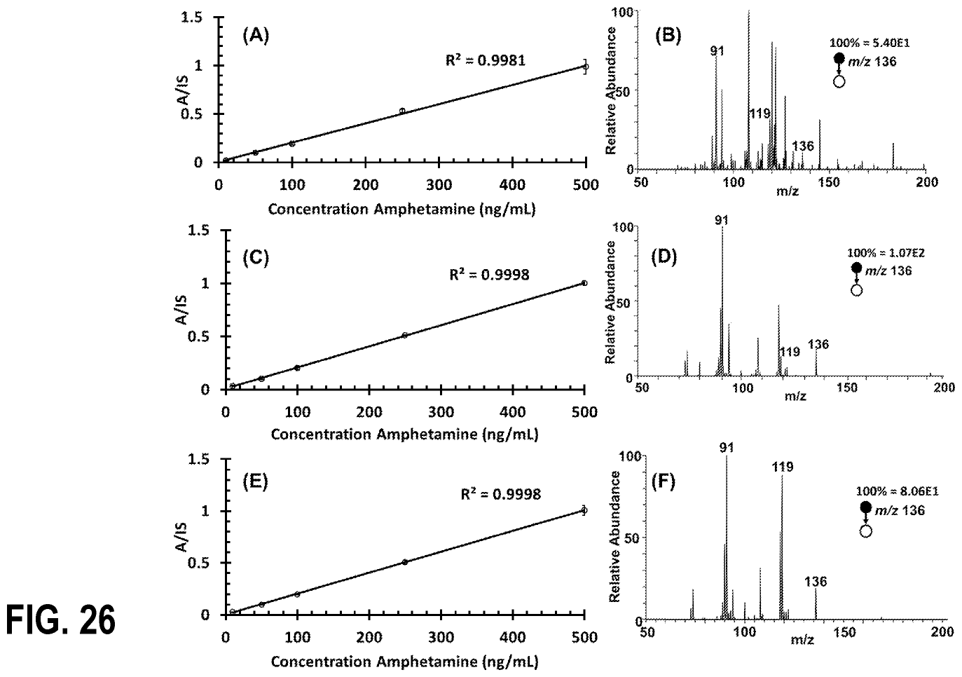

Quantification was performed by spiking illicit drugs and their internal standards into human whole blood at concentrations ranging from 10 to 500 ng/mL, and 4 μL dried blood spheroids were analyzed using PS MS with 20 μL ethyl acetate spray solvent. Not only did the ion signal last approximately twice as long when hydrophobic paper (30 and 120 min treated paper) was used, but limits of detection (LODs) as low as 0.12 ng/mL (corresponding to 10× reduction in LOD and LOQ for amphetamine on 30 min treated paper), were observed including a more linear response to concentration (R2>0.999; FIG. 23-26).

Whole human blood was spiked with 10, 50, 100, 250, and 500 ng/mL of cocaine, benzoylecgonine, amphetamine, and methamphetamine separately. 4 μL of blood was deposited onto untreated paper triangles and paper triangles treated with silane for 30 minutes and 2 hours. The blood spots were allowed to dry for 24 hours. 3 kV was applied to the paper triangles, and 20 μL ethyl acetate was applied to the triangle. Quantification of each drug was performed by analyzing the main fragment from each drug: cocaine (304→182), benzoylecgonine (290→168), methamphetamine (150→119) and amphetamine (136→119). Resulting limits of detection and limits of quantification are summarized in Table 4 (above).

Compared with untreated paper substrates, the lower LODs calculated for hydrophobic paper are mainly attributed to: i) the inability of the blood sample to wet through the paper—the fact that the aqueous-based blood samples are unable to wet through the fiber core of the hydrophobic paper and spread suggests interactions between drug and the paper surface prior to extraction is decreased. This results in a greater number of free drug analytes available in the dried spheroid, increasing analyte signal; ii) the more uniform spot size for the dried spheroids—this contributes to the observed quantitative abilities (i.e., lower LODs and improved linearity) by creating a more reproducible extraction area and decreasing variations in analyte signal; and iii) the decreased analyte binding capacity to the paper post extraction That is, redistribution of extracted analyte back into the hydrophobic paper is reduced compared with hydrophilic paper substrates.

In summary, by using a hydrophobic paper substrate, the present inventors have established a dried blood spheroid collection platform that has potential of eliminating chromatographic/volcanic effects associated with the traditional dried blood spot samples. The dried blood spheroid sample collection platform showed increased stability for hydrolytically labile compounds against oxidative stress, increasing the lifetime of diazepam, cocaine and benzoylecgonine (the main metabolite of cocaine), from days to several weeks under ambient conditions and without cold storage. Through manipulation of the surface energy of the paper and the use of organic spray solvent (e.g., ethyl acetate immiscible with blood and reducing matrix effects), selective extraction of target analytes may be performed, which allows enhanced PS MS detection of cocaine, benzoylecgonine, amphetamine, and methamphetamine from the dried blood spheroids, resulting in sub ng/mL limits of detection. Manipulation of solvent surface tension allows determination of surface energy of the porous hydrophobic paper substrate without contact angle measurements. This novel electrostatic method employed a simple multimeter detector. Because of its close resembles DBS, the implementation of dried blood spheroid sample collection in clinical settings can be accomplished with no changes in blood collection procedures.

### Example 2: Synthesis of Novel Probes as Mass Reporters for MS-Based Immunoassays

This Example describes the design and synthesis of chemical probes with the capacity to generate reported compounds upon stimulation. Stimuli include pH change and UV-light illumination. The probes have three functional properties: (1) isothiocyanate (—NCS) or N-hydroxysuccinimide (NHS) groups for coupling to antibodies, (2) a charge-labeled quaternary ammonium species (QUAT), which has a stable positive charge, for sensitive detection by MS, and (3) a cleavable linker for release of the probe from the bound antibody.

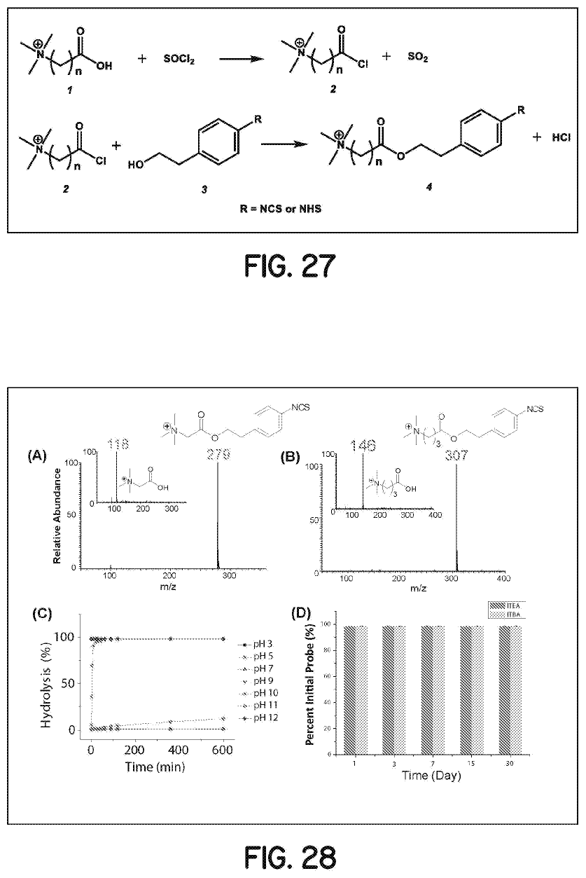

pH-sensitive ionic probes with an ester functional group was used as the pH cleavable bond because (1) it is highly stable at neutral conditions, and (2) compared with other cleavable modes (e.g. photocleavage, oxidation, or reduction), changing solution pH to release the bound ion is simpler. A synthetic procedure for making the pH sensitive ionic probes is as shown in FIG. 27. This synthetic approach was designed based on commercially available starting materials. In the first step, thionyl chloride (SOCl₂) converts the carboxylic acid group in 1 into an acid chloride 2, having more reactivity towards 3 to give the desired product 4. Using this approach, two new pH-sensitive ionic probes, 2-(4-isothiocyanatophenethoxy)-N,N,N-trimethyl-2-oxoethanaminium chloride (ITEA, n=1) and 4-(4-isothiocya-natophenethoxy)-N,N,N-trimethyl-4-oxobutan-1-aminiumchloride (ITBA, n=3), have been synthesized as mass reporters. The two probes differ only in the distance (n) between the QUAT and the ester cleavable bond. The intact molecular ions of ITEA and ITBA are detected by MS at m/z 279 and m/z 307, respectively (see FIG. 28 panels A, B); the respective QUAT charge-tags (m/z 118 from ITEA and m/z 146 from ITBA; see FIG. 28, inserts) are easily released in the presence of basic NH₄OH solution (FIG. 28, panel C). Both probes are stable under neutral conditions (pH 7) even after 30 days of storage (FIG. 28, panel D).

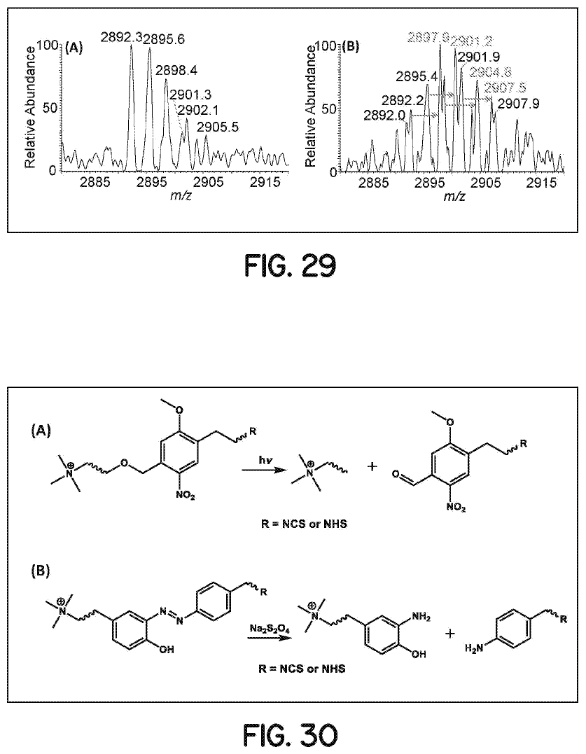

The synthesized ionic probes were then coupled to anti-histidine-rich protein-II(HRP-II) antibodies (FIG. 29) in which the coupling efficiencies were: 2 copies of ITBA per antibody and 1 copy of ITEA per antibody. These conjugates have also been used for malaria detection (detailed in Example 3, below) and detection limits (LoDs) were 75 pM (2.8 ng/mL) and 1 nM (37 ng/mL) for ITBA and ITEA ionic probes, respectively.

The proximity (indicated by carbon chain (n)) of the electron-withdrawing QUAT cation to the ester bond has been found to influence the hydrolysis rate of the probes. The design with longer carbon chain can improve (a) coupling efficiency, (b) the stability of the probe during test storage, and (c) sensitivity—the longer linear chain will make it easier to be fragmented in collision-induced dissociation (CID) during MS/MS analysis and hence producing signature productions with higher efficiency and intensity.

Photo-cleavable ionic probes: Due to their pH insensitivity, photo-cleavable probes are expected to offer much higher coupling efficiency at pH 9. In general, the strategy of photo-cleavage is frequently used in biochemical research because it is rapid, efficient, specific, and provides clean reaction system with no ion suppression effects during MS analysis. Due to their rapid photo-reactivity (photo-cleavage in the near-UV at 330-370 nm), o-nitrobenzyl derivatives have become one of the most popular photo-labile molecules. To adopt these compounds to an MS-based immunoassay, we can incorporate a charged reporter group (i.e., QUAT) and reactive labeling group (e.g. NCS or NHS) into the o-nitrobenzyl unit (FIG. 30, panel A). Again, the NCS/NHS functional groups will be used for coupling of the probe to the antibody.

Redox chemistry: The third cleavable reaction of interest will involve the redox chemistry of diazobenzenes (FIG. 30, panel B). Diazobenzene motifs are cleavable via reduction, for instance with sodium dithionite (Na₂S₂O₄) under mild reaction conditions.

The present inventors applied this procedure for the detection of PfHRP-2 malaria antigen from undiluted human serum. As already discussed (see Example 2, above), the LoDs of this experiment were 75 pM (2.8 ng/mL) and 1 nM (37 ng/mL) for ITBA and ITEA ionic probes, respectively (FIG. 34, panel B). The corresponding absolute amounts were 1.5 fmol/zone and 50 fmol/zone, respectively. This sensitivity is comparable to that of the enzyme amplified methods recorded in our hands when using similar antibodies (ELISA LoD: 1 ng/mL for PfHRP-2 in serum), although no amplification is adopted for our MS-based method. These results indicate that the MS immunoassay can be used to diagnose malaria infection for blood parasite densities of 200 parasites μL⁻¹ (mean antigen concentration is 9.1 ng/mL), which is the WHO recommended lowest density for diagnosis.

To enable surveillance testing, the proposed paper-based immunoassay test needs to be stable and robust. The paper device containing the captured malaria PfHRP-2 antigen can be stored for at least 30 days (at room temperature) without affecting test results (FIG. 34, panel C). This is in contrast to enzyme amplified tests where assay signal dropped to zero after the test was stored under dry conditions for just 2 h (red line, FIG. 34, panel D). In buffer solutions, the signal dropped to 23% of the initial value after 7 days of storage (black line, FIG. 34, panel D). Collectively, these experiments demonstrated that by using the disclosed MS immunoassay protocol, two separate end-points can be provided in which the assay can be interrupted, stored and restored.

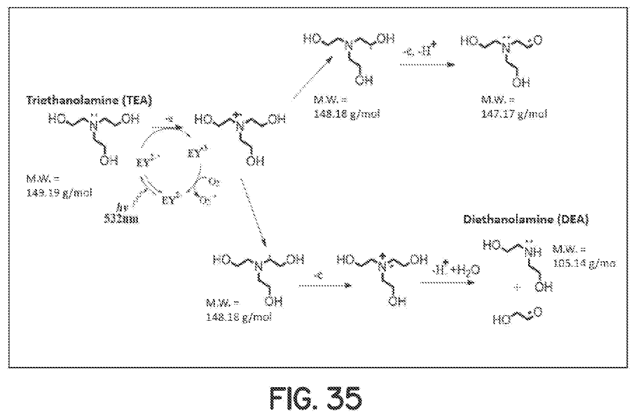

Coupling a photo-catalyst to the reporter antibody permits generation of a reporter compound without needing a cleavable group. (FIG. 32). Unlike enzyme amplification, this amine-based MS amplification process can be terminated by removing the light source therefore enabling test analysis at a later convenient time without affecting diagnostic outcome. Eosin Y (EY) was chosen as the photo-catalyst, for reaction with ethanolamine. Triethanolamine (TEA) was selected as substrate for this photo-redox reaction due to its usefulness as a sacrificial electron donor in a three-component system (TCS). The TCS consists of TEA as the sacrificial electron donor (SD), Eosin Y, an organodye, as the photosensitizer (PS) and oxygen, which serves as the final electron donor to regenerate the photo-catalyst. This chemistry is illustrated in FIG. 35, which can produce two distinct final products (molecular weights 147 and 105 g/mol).

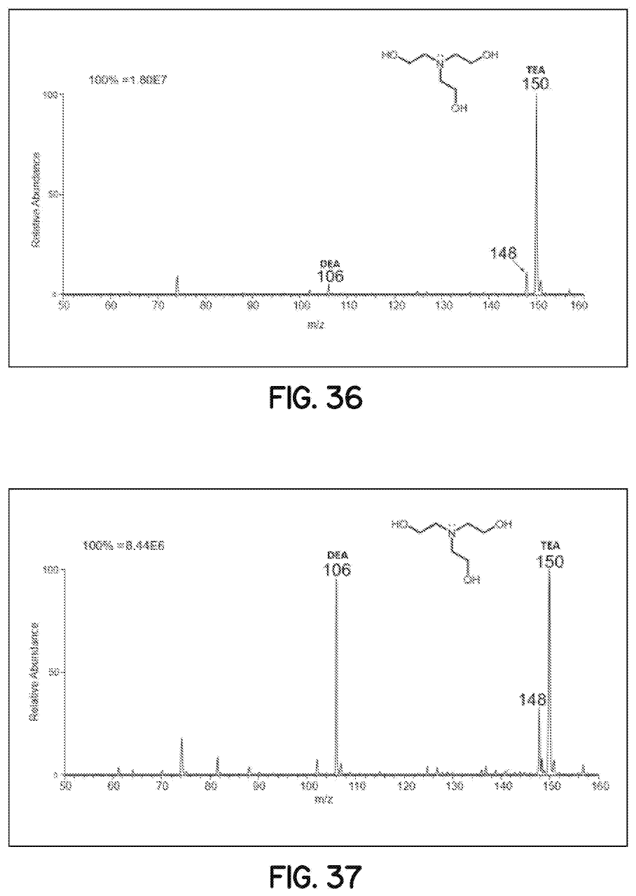

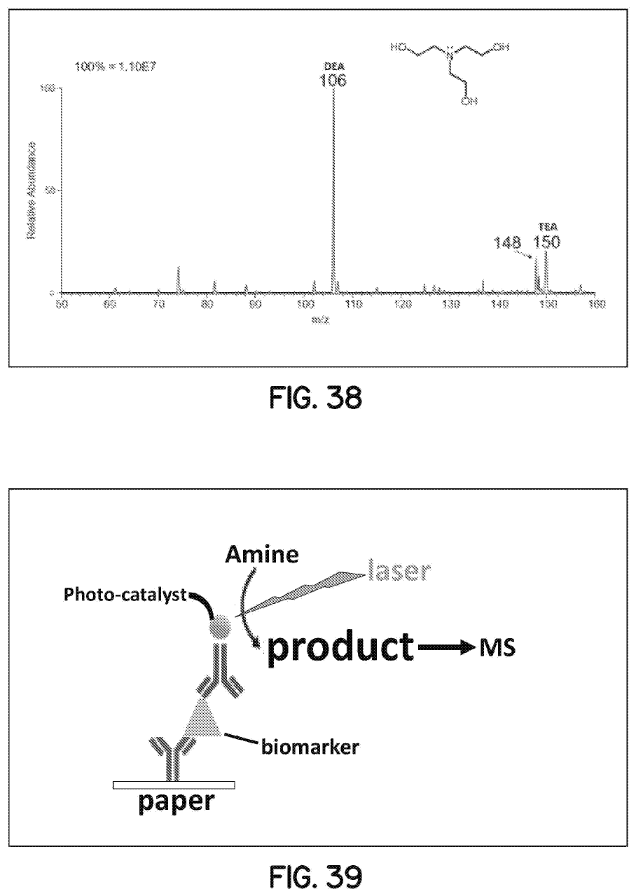

To test this possible three component photocatalytic system toward MS signal amplification, 1 ppm solution of TEA was prepared containing catalytic amounts of 25 μM EY. Real-time analysis was carried to monitor the possible reaction products ensuing from this eosin-based photo-catalysis. The spectra for this analysis are summarized in FIGS. 36-38. Results from this experiment indicate that ethanolamine (m/z 150) can be catalytically converted to (m/z 106) diethanolamine in less than 2 minutes of visible light illumination (FIG. 37). The change in mass (m/z) will serve a positive signal indicating the presence of a disease biomarker. Since eosin is regenerated in the process, during the process, one will be able to use large quantities of the ethanolamine substrate that will serve to amplify detection of few biomarkers captured by the antibody, which is conjugated to eosin (FIG. 39).

The amount of TEA decreases with illumination time, from FIGS. 36 to 38, while the intensity of product DEA increases with reaction time. This means the detection of DEA at m/z 106 when TEA is added to test zone will signify the presence of eosin, which can only be captured if biomarker is bound to paper.

### Example 3: Biomarkers for CRC-Specific Blood Testing

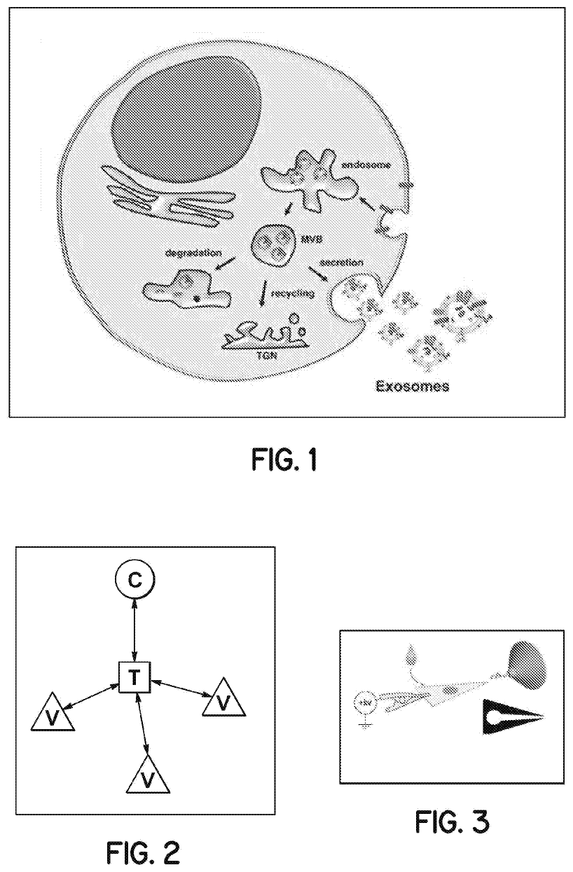

Carcinoembryonic antigen (CEA) is often used as a biomarker for screening of CRC. However, CEA is not ideal due to its low specificity (positive detection rate is ˜45-60%) as it is detected in almost all gastrointestinal tumors. Multiplexing other biomarkers with CEA can improve the positive detection rate for detecting CRC cancer. Therefore, the present inventors selected exosomal biomarker A33 in addition to CEA. Exosomes (FIG. 1) mediate cell-to-cell communication by transferring bioactive molecules such as proteins, lipids, RNAs, and mitochondrial DNA, some of which are exosome inherent, and some of which represent their cells of origin. While exosomes are secreted by multiple cell types, cancer derived exosomes not only influence the invasive potentials of proximally located cells, but also affect distantly located tissues. Proteomic studies have identified A33 as a 43-kDa membrane-bound glycoprotein present on the basolateral surface of normal colon and small bowel epithelial cells, which is homogenously expressed in 95% of human colorectal cancers but not in most other tumor types or non-gastrointestinal tract tissues.

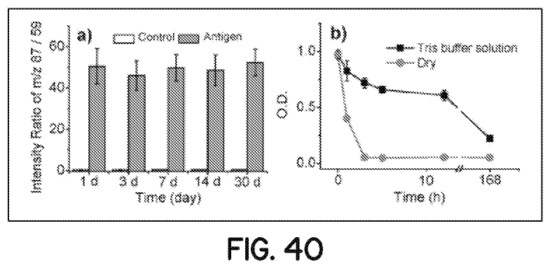

Two cleavable ionic probes have been designed and synthesized. The present inventors have coupled the synthesized probes to anti-PfHRP-2 antibodies, and used these conjugates for malaria diagnosis on paper [detection limit (LoD) is 2.8 ng/mL ng/mL in serum]. The stability of the paper device after PfHRP-2 capture has also been investigated and found to yield a more stable signal when compared with enzyme-amplified detection (FIG. 40).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method of detecting at least one antigen in a biological sample, comprising:
a) contacting the biological sample with a hydrophobic cellulose substrate comprising a capture antibody;
b) contacting the substrate with a reporter antibody to form a capture antibody-antigen-reporter antibody sandwich complex;
c) washing the substrate to remove unbound reporter antibody;
d) reacting the reporter antibody to generate a reporter compound; and
e) detecting the reporter compound using mass spectrometry.

2. The method of claim 1, wherein the reporter compound comprises a quaternary amine or a secondary amine.

3. (canceled)

4. The method of claim 1, wherein the reporter compound is conjugated to the reporter antibody through a selectively cleavable linker, and the reporter compound is generated by cleaving said linker.

5. (canceled)

6. The method of claim 1, wherein the reporter antibody comprises a photoredox catalyst, and the reporter compound is generated by reacting a sacrificial electron donor with the photoredox catalyst in the presence of light and oxygen.

7. The method of claim 6, wherein the sacrificial electron donor comprises a tertiary amine.

8. The method of claim 1, wherein the reporter antibody is conjugated to a gold nanoparticle.

9. The method of claim 8, wherein the gold nanoparticle is conjugated to the reporter compound.

10. The method of claim 8, wherein the gold nanoparticle is conjugated to a photoredox catalyst.

11-13. (canceled)

14. The method of claim 1, wherein the hydrophobic cellulose substrate includes a base and first and second edges, the first and second edges intersect the base at first ends thereof, and a sum of the angles at which the first and second edges intersect the base is greater than 135 degrees.

15. The method of claim 14, wherein the substrate comprises a fluid-impermeable barrier permeating a thickness of the substrate, and the substrate defines a boundary of a reservoir region and a boundary of a channel region, the reservoir region and the channel region being in fluid communication with each other, the channel region extending between second ends of the first and second edges and the reservoir, the second ends of the first and second edges being opposite the first ends.

16. (canceled)

17. An assay device comprising a plurality of porous layers, comprising:
a) a reagent layer comprising a reporter antibody disposed in a reporter region;
b) a capture layer comprising a capture antibody disposed in a capture region, wherein said capture antibody is conjugated to the porous substrate of the capture layer;
c) a detection layer comprising a hydrophobic cellulose substrate;
wherein the reporter region is in fluid communication with the capture region, and the detection layer is in fluid communication with the capture region.

18. (canceled)

19. The device of claim 17, wherein the hydrophobic cellulose substrate in the detection layer includes a base and first and second edges, the first and second edges intersect the base at first ends thereof, and a sum of the angles at which the first and second edges intersect the base is greater than 135 degrees.

20. The device of claim 19, wherein the hydrophobic cellulose substrate in the detection layer comprises a fluid-impermeable barrier permeating a thickness of the substrate, and the substrate defines a boundary of a reservoir region and a boundary of a channel region, the reservoir region and the channel region being in fluid communication with each other, the channel region extending between second ends of the first and second edges and the reservoir, the second ends of the first and second edges being opposite the first ends.

21. The device of claim 17, further comprising a dwell layer disposed between the reagent layer and capture layer.

22. The device of claim 17 further comprising a plasma separation layer disposed upstream of the reagent layer.

23. The device of claim 17, comprising a fluid impermeable barrier permeating the thickness of the reporter layer and the thickness of the capture layer, said barrier defining the boundary of the reporter region, the boundary of the capture region, and the boundary of a channel fluidically connecting the reagent region and the capture region.

24. The device of claim 23, wherein the reagent layer comprises a plurality of reagent regions defined by the boundaries of the fluid impermeable barrier permeating the reagent layer, and
further comprising a splitter layer disposed upstream of the reagent layer,
wherein the splitter layer comprises a fluid impermeable barrier permeating its thickness, said barrier defining the boundary of a central splitter reservoir, the boundaries of a plurality of peripheral splitter reservoirs disposed peripherally relative to the central splitter reservoir and spaced apart therefrom, and a plurality of channels, each channel fluidically connecting a respective peripheral splitter reservoir with the central splitter reservoir;
wherein each peripheral splitter reservoir is fluidically connected to one reagent region.

25. The device of claim 23, wherein the capture layer comprises a plurality of capture regions defined by the boundaries of the fluid impermeable barrier permeating the capture layer, and
further comprising a collimater layer disposed between the capture layer and detection layer, said collimater layer comprising a fluid impermeable barrier permeating its thickness, said barrier defining a central collimater reservoir, the boundaries of a plurality of peripheral collimator reservoirs disposed peripherally relative to the central collimater reservoir and spaced apart therefrom, and a plurality of channels, each channel fluidically connecting a respective peripheral collimator reservoir with the central collimator reservoir,
wherein each peripheral collimator reservoir is fluidically connected to one capture region; and
wherein the central collimator reservoir is fluidically connected to the detector layer.

26. The device of claim 17, further comprising a removable hydrophobic barrier layer disposed between the capture layer and the detector layer, between the capture layer and the collimating layer, or between both the capture layer and the detector layer and the capture layer and the collimating layer.

27. (canceled)

28. A substrate for paper spray mass spectrometry, comprising a triangular shaped hydrophobic cellulose substrate, the substrate includes a base and first and second edges, the first and second edges intersect the base at first ends thereof, and a sum of the angles at which the first and second edges intersect the base is greater than 135 degrees, wherein the substrate comprises silane-functionalized cellulose, and wherein after the substrate is contacted with biological sample and dried, the resulting dried sample is in the shape of a spheroid, wherein the distance from the surface of the substrate to the highest point in the spheroid is at least 50% the diameter of the spheroid.

29-37. (canceled)
